(19) United States
(12) Patent Application Publication
(10) Pub. No.: US 2016/0238590 A1
(43) Pub. Date: Aug. 18, 2016

(54) COMPOSITIONS AND METHODS RELATED TO OAT SENSITIVITY

(71) Applicant: IMMUSANT, INC., Cambridge, MA (US)

(72) Inventors: Robert P. Anderson, Shrewsbury, MA (US); Jason A. Tye-Din, Collingwood (AU); Melinda Hardy, Brunswick West (AU)

(21) Appl. No.: 15/022,952

(22) PCT Filed: Sep. 20, 2013
(86) PCT No.: PCT/US13/60939

§ 371 Date: Mar. 18, 2016

(51) Int. Cl. G01N 33/50; C07K 16/16; G01N 33/564; A61K 39/35

(52) U.S. Cl. CPC G01N 33/5014; A61K 39/35; C07K 16/16; G01N 33/505; G01N 33/564; A23L 1/293; A61K 2039/577

(57) ABSTRACT

Provided herein are methods, kits and compositions related to identifying or treating a subject sensitive to or likely to be sensitive to oats.

### BACKGROUND

Celiac disease, also known as coeliac disease or Celiac sprue (Coeliac sprue), affects approximately 1% of people in Europe and North America. Celiac disease occurs in genetically susceptible individuals who possess either HLA-DQ2.5 (encoded by the genes HLA-DQA1*05 and HLA-DQB1*02) accounting for about 90% of individuals, HLA-DQ2.2 (encoded by the genes HLA-DQA1*02 and HLA-DQB1*02), or HLA-DQ8 (encoded by the genes HLA-DQA1*03 and HLA-DQB1*0302). Without wishing to be bound by theory, it is believed that such individuals mount an inappropriate HLA-DQ2- and/or DQ8-restricted CD4⁺ T cell-mediated immune response to peptides derived from the aqueous-insoluble proteins of wheat flour, gluten, and related proteins in rye and barley.

A gluten free diet is the only currently approved treatment for Celiac disease. Many patients are non-compliant to this diet due to the lack of choice and low palatability. Patients would benefit from the addition of oats to their diet, but there is some evidence that oats are also toxic to some CD patients.

### SUMMARY

Described herein are compositions, kits and methods related to identifying and/or treating a subject sensitive to or likely to be sensitive to oats.

In one aspect, a method for identifying a subject as sensitive to or likely sensitive to oats is provided. In one embodiment, the method comprising determining a T cell response to a barley peptide in a sample comprising a T cell from the subject; and identifying whether or not the subject is sensitive or likely sensitive to oats. In some embodiments, the method of identifying comprises comparing a T cell response to the barley peptide to a control T cell response. In some embodiments, the subject is sensitive to or likely sensitive to oats if the T cell response to the barley peptide is elevated compared to a control T cell response, or not sensitive to or likely not sensitive to oats if the T cell response to the barley peptide is reduced compared to the control T cell response or the same as the control T cell response.

In some embodiments, the step of determining comprises contacting the sample with a composition comprising the barley peptide and measuring a T cell response to the barley peptide. In some embodiments, measuring a T cell response to the barley peptide comprises measuring a level of IFN-gamma in the sample. In some embodiments, measuring comprises an enzyme-linked immunosorbent assay (ELISA) or an enzyme-linked immunosorbent spot (ELISpot) assay.

In some embodiments, the barley peptide is a hordein peptide. In some embodiments, the hordein peptide comprises any of the hordein peptides provided herein. In some embodiments, the hordein peptide comprises the amino acid sequence PIPQQPQPY (SEQ ID NO: 1), PYPQQPQPY (SEQ ID NO: 2), PFPQQPQPY (SEQ ID NO: 3), PIPEQPQPY (SEQ ID NO: 4), PYPEQPQPY (SEQ ID NO: 5), PFPEQPQPY (SEQ ID NO: 6), PIPDQPQPY (SEQ ID NO: 7), PYPDQPQPY (SEQ ID NO: 8), or PFPDQPQPY (SEQ ID NO: 9). In some embodiments, the hordein peptide comprises the amino acid sequence PIPQQPQPY (SEQ ID NO: 1), PIPEQPQPY (SEQ ID NO: 4), or PIPDQPQPY (SEQ ID NO: 7). In some embodiments, the hordein peptide comprises the amino acid sequence PIPEQPQPY (SEQ ID NO: 4). In some embodiments, the hordein peptide comprises PYPEQPQPF (SEQ ID NO: 10).

In some embodiments, the subject has Celiac disease.

In some embodiments, the method further comprises performing an additional test if the subject is identified as sensitive to or likely sensitive to oats. In some embodiments, the additional test comprises measuring a T cell response to an oat peptide. In some embodiments, a sample comprising a T cell from the subject is contacted with a composition comprising the oat peptide. In some embodiments, the oat peptide comprises any of the oat peptides provided herein. In some embodiments, the oat peptide comprises the amino acid sequence PYPEQQQPI (SEQ ID NO: 11), PYPEQEQPI (SEQ ID NO: 12), PYPEQDQPI (SEQ ID NO: 13), PYPDQEQPI (SEQ ID NO: 14) or PYPDQDQPI (SEQ ID NO: 15). In some embodiments, the oat peptide comprises the amino acid sequence PYPEQEQPI (SEQ ID NO: 12). In some embodiments, the oat peptide comprises: the amino acid sequence of Genbank AAB32025 (8-21) YQPYPEQQQPILQQ (SEQ ID NO: 16) or its partially deamidated homolog Genbank AAB32025 (8-21) [Q15 to E] YQPYPEQEQPILQQ (SEQ ID NO: 17); the amino acid sequence of Genbank AAA32714.1 (25-40) EQYQPYPEQQPFMQPL (SEQ ID NO: 18), Genbank AAB23365.1 (3-18) TVQYDPSEQYQPYPEQ (SEQ ID NO: 19) or Genbank AAA32716.1 (20-39) TTTVQYNPSEQYQPYPEQQE (SEQ ID NO: 20) or the partially deamidated homolog Genbank AAA32714.1 (25-40) [Q32 to E] EQYQPYPEEQPFMQPL (SEQ ID NO: 21) or Genbank AAA32716.1 (20-39)[Q38 to E] TTTVQYNPSEQYQPYPEQEE (SEQ ID NO: 22); the amino acid sequence of Genbank AAB23365.1 (9-24) SEQYQPYPEQQQPFVQ (SEQ ID NO: 23) or Genbank Q09097.1 (1-20) TTTVQYDPSEQYQPYPEQQE (SEQ ID NO: 24) or the partially deamidated homolog Genbank AAB23365.1 (9-24) [Q19 to E] SEQYQPYPEQEQPFVQ (SEQ ID NO: 25); the amino acid sequence of Genbank AAB32025 (7-22) QYQPYPEQQQPILQQQ (SEQ ID NO: 26) or its partially deamidated homolog Genbank AAB32025 (7-22) [Q15 to E] QYQPYPEQEQPILQQQ (SEQ ID NO: 27); the amino acid sequence of Genbank AAA32715.1 (19-38) AQFDPSEQYQPYPEQQQPIL (SEQ ID NO: 28), Genbank AAB23365.1 (10-29) EQYQPYPEQQQPFVQQQPPF (SEQ ID NO: 29), Genbank P14812.1 (402-421) NNHGQTVFNDILRRGQLLII (SEQ ID NO: 30) or Genbank Q09095.1 (3-22) EQYQPYPEQQQPFLQQQPLE (SEQ ID NO: 31) or the partially deamidated Genbank Q09097.1 (9-28) [Q19 to E] SEQYQPYPEQEEPFVQQQPP (SEQ ID NO: 32), Genbank Q09095.1 (2-21) [Q12 and Q18 to E] SEQYQPYPEQEQPFLQEQPL (SEQ ID NO: 33), Genbank AAB23365.1 (10-29) [Q19 to E] EQYQPYPEQEQPFVQQQPPF (SEQ ID NO: 34) or Genbank AAB32025 (4-23) [Q15, Q22, and Q23 to E] PSEQYQPYPEQEQPILQQEE (SEQ ID NO: 35).

In some embodiments, the sample comprises whole blood or peripheral blood mononuclear cells.

In some embodiments, the method further comprises administering a composition comprising barley or oats, or a peptide thereof, to the subject prior to determining the T cell response. In some embodiments, the composition comprising barley or oats, or a peptide thereof, is administered to the subject more than once prior to determining the T cell response. In some embodiments, the composition comprising barley or oats, or a peptide thereof, is a foodstuff. In some embodiments, the composition comprises a barley peptide. In some embodiments, the barley peptide is a hordein peptide. In some embodiments, the hordein peptide comprises any of the hordein peptides provided herein.

In some embodiments, the method further comprises treating the subject if identified as sensitive or likely sensitive to oats or providing information to the subject about a treatment. In some embodiments, the method further comprises a step of recommending an oats-free diet if the subject is identified as sensitive to or likely sensitive to oats or providing information to the subject about such a diet.

In one aspect, a method for identifying a subject as sensitive to or likely sensitive to oats is provided, wherein the method comprises determining a T cell response to any of the oat peptides provide herein in a sample comprising a T cell from the subject; and identifying whether or not the subject is sensitive to or likely sensitive to oats. In some embodiments, the peptide comprises the amino acid sequence PYPEQQQPI (SEQ ID NO: 11), PYPEQEQPI (SEQ ID NO: 12), PYPEQDQPI (SEQ ID NO: 13), PYPDQEQPI (SEQ ID NO: 14) or PYPDQDQPI (SEQ ID NO: 15). In some embodiments, the peptide comprises the amino acid sequence PYPEQEQPI (SEQ ID NO: 12). In some embodiments, identifying comprises comparing the T cell response to the peptide to a control T cell response. In some embodiments, the subject is sensitive to or likely sensitive to oats if the T cell response to the peptide is elevated compared to a control T cell response, or not sensitive to or likely not sensitive to oats if the T cell response to the peptide is reduced compared to the control T cell response or the same as the control T cell response.

In some embodiments, the step of determining comprises contacting the sample with a composition comprising the peptide and measuring a T cell response to the peptide. In some embodiments, measuring a T cell response to the peptide comprises measuring a level of IFN-gamma in the sample. In some embodiments, measuring comprises an enzyme-linked immunosorbent assay (ELISA) or an enzyme-linked immunosorbent spot (ELISpot) assay.

In some embodiments, the subject has Celiac disease.

In some embodiments, the sample comprises whole blood or peripheral blood mononuclear cells.

In some embodiments, the method further comprises administering a composition comprising barley or oats, or a peptide thereof, to the subject prior to determining the T cell response. In some embodiments, the composition comprising barley or oats, or a peptide thereof, is administered to the subject more than once prior to determining the T cell response. In some embodiments, the composition comprising barley or oats, or a peptide thereof, is a foodstuff. In some embodiments, the composition comprises a barley peptide. In some embodiments, the barley peptide is a hordein peptide. In some embodiments, the hordein peptide comprises any of the hordein peptides provided herein.

In some embodiments, the method further comprises treating the subject if identified as sensitive or likely sensitive to oats or providing information to the subject about a treatment. In some embodiments, the method further comprises a step of recommending an oats-free diet if the subject is identified as sensitive to or likely sensitive to oats or providing information to the subject about such a diet.

In one aspect a composition comprising any of the peptides provided herein is provided. In some embodiments, the peptide comprises the amino acid sequence PYPEQQQPI (SEQ ID NO: 11), PYPEQEQPI (SEQ ID NO: 12), PYPEQDQPI (SEQ ID NO: 13), PYPDQEQPI (SEQ ID NO: 14) or PYPDQDQPI (SEQ ID NO: 15). In some embodiments, the peptide comprises the amino acid sequence YQPYPEQQQPILQQ (SEQ ID NO: 16), YQPYPEQEQPILQQ (SEQ ID NO: 17), YQPYPEQDQPILQQ (SEQ ID NO: 36), YQPYPDQEQPILQQ (SEQ ID NO: 37) or YQPYPDQDQPILQQ (SEQ ID NO: 38). In some embodiments, the peptide comprises the amino acid sequence PYPEQEQPI (SEQ ID NO: 12). In some emboidments, the peptide comprises the amino acid sequence YQPYPEQEQPILQQ (SEQ ID NO: 17). In some embodiments, the peptide comprises the Genbank AAB32025 (8-21) YQPYPEQQQPILQQ (SEQ ID NO: 16) or its partially deamidated homolog Genbank AAB32025 (8-21) [Q15 to E] YQPYPEQEQPILQQ (SEQ ID NO: 17); the amino acid sequence of Genbank AAA32714.1 (25-40) EQYQPYPEQQPFMQPL (SEQ ID NO: 18), Genbank AAB23365.1 (3-18) TVQYDPSEQYQPYPEQ (SEQ ID NO: 19) or Genbank AAA32716.1 (20-39) TTTVQYNPSEQYQPYPEQQE (SEQ ID NO: 20) or the partially deamidated homolog Genbank AAA32714.1 (25-40) [Q32 to E] EQYQPYPEEQPFMQPL (SEQ ID NO: 21) or Genbank AAA32716.1 (20-39)[Q38 to E] TTTVQYNPSEQYQPYPEQEE (SEQ ID NO: 22); the amino acid sequence of Genbank AAB23365.1 (9-24) SEQYQPYPEQQQPFVQ (SEQ ID NO: 23) or Genbank Q09097.1 (1-20) TTTVQYDPSEQYQPYPEQQE (SEQ ID NO: 24) or the partially deamidated homolog Genbank AAB23365.1 (9-24) [Q19 to E] SEQYQPYPEQEQPFVQ (SEQ ID NO: 25); the amino acid sequence of Genbank AAB32025 (7-22) QYQPYPEQQQPILQQQ (SEQ ID NO: 26) or its partially deamidated homolog Genbank AAB32025 (7-22) [Q15 to E] QYQPYPEQEQPILQQQ (SEQ ID NO: 27); the amino acid sequence of Genbank AAA32715.1 (19-38) AQFDPSEQYQPYPEQQQPIL (SEQ ID NO: 28), Genbank AAB23365.1 (10-29) EQYQPYPEQQQPFVQQQPPF (SEQ ID NO: 29), Genbank P14812.1 (402-421) NNHGQTVFNDILRRGQLLII (SEQ ID NO: 30) or Genbank Q09095.1 (3-22) EQYQPYPEQQQPFLQQQPLE (SEQ ID NO: 31) or the partially deamidated Genbank Q09097.1 (9-28) [Q19 to E] SEQYQPYPEQEEPFVQQQPP (SEQ ID NO: 32), Genbank Q09095.1 (2-21) [Q12 and Q18 to E] SEQYQPYPEQEQPFLQEQPL (SEQ ID NO: 33), Genbank AAB23365.1 (10-29) [Q19 to E] EQYQPYPEQEQPFVQQQPPF (SEQ ID NO: 34) or Genbank AAB32025 (4-23) [Q15, Q22, and Q23 to E] PSEQYQPYPEQEQPILQQEE (SEQ ID NO: 35). In some embodiments, the peptide is less than 50 amino acids in length. In some embodiments, the peptide is less than 30 amino acids in length.

In one aspect, a vaccine composition comprising any of the compositions provided herein is provided.

In one aspect, a kit comprising any of the compositions provided herein is provided. In some embodiments, the kit comprises a container for whole blood. In some embodiments, the composition is dried on the wall of the container for whole blood. In some embodiments, the kit comprises a negative control container. In some embodiments, the kit comprises a positive control container. In some embodiments, the negative and/or positive control container(s) are present in duplicate or triplicate. In some embodiments, any or all of the containers can be a vial or tube. In some embodiments, the composition is in solution or lyophilized in a separate container.

In one aspect, a method of modulating a T cell response to an oat peptide in a subject who is sensitive to oats, the method comprising administering to the subject an effective amount of a composition comprising any of the oat peptides provided herein is provided. In some embodiments, the peptide comprises the amino acid sequence PYPEQQQPI (SEQ ID NO: 11), PYPEQEQPI (SEQ ID NO: 12), PYPEQDQPI (SEQ ID NO: 13), PYPDQEQPI (SEQ ID NO: 14) or PYPDQDQPI (SEQ ID NO: 15). In some embodiments, the peptide comprises the amino acid sequence PYPEQQQPI (SEQ ID NO: 11) or PYPEQEQPI (SEQ ID NO: 12). In some embodiments, the peptide is less than 50 amino acids in length. In some embodiments, the peptide is less than 30 amino acids in length. In some embodiments, the peptide comprises the amino acid sequence YQPYPEQQQPILQQ (SEQ ID NO: 16), YQPYPEQEQPILQQ (SEQ ID NO: 17), YQPYPEQDQPILQQ (SEQ ID NO: 36), YQPYPDQEQPILQQ (SEQ ID NO: 37) or YQPYPDQDQPILQQ (SEQ ID NO: 38). In some embodiments, the peptide comprises the amino acid sequence YQPYPEQQQPILQQ (SEQ ID NO: 16) or YQPYPEQEQPILQQ (SEQ ID NO: 17).

In one aspect, a method, comprising detecting the presence of any of the oat peptides provided herein in a composition is provided. In some embodiments, the peptide comprises the amino acid sequence PYPEQEQPI (SEQ ID NO: 12) and/or PYPEQQQPI (SEQ ID NO: 11). In some embodiments, the method is for determining whether the composition is capable of causing a T cell response in a subject. In some embodiments, the composition is a foodstuff.

In one aspect, an antibody that specifically binds to any of the peptides provided herein is provided.

In another aspect, the use of any of the peptides provided herein for producing an antibody that specifically binds to the peptide is provided.

In any of the methods, kits or compositions provided herein the peptide can comprise any of the peptides provided herein.

In any of the methods, kits or compositions provided herein the peptide can be the wild-type version, a deamidated version or an aspartate-substituted version.

In any of the methods, kits or compositions provided herein the peptide is modified such that an N-terminal glutamate or glutamine is substituted with a pyroglutamate residue and the C-terminal carboxyl group of the peptide is amidated. In any of the methods, kits or compositions provided herein the peptide is modified such that it further comprises an N-terminal pyroglutamate residue and the C-terminal carboxyl group of the peptide is amidated.

### BRIEF DESCRIPTION OF THE DRAWINGS

FIG. 1 is a series of graphs showing induction of DQ2.5-ave-1c(PYPEQEQPI, SEQ ID NO: 12)-specific T cell responses in patients following 3-day barley challenge. HLA-DQ2.5⁺ CD patients undertook a 3-day combined wheat, rye, and barley challenge or a barley challenge and on day 6 PBMC were tested against both the barley peptide containing DQ2.5-hor-3a (PIPEQPQPY, SEQ ID NO: 4) and the avenin peptide containing DQ2.5-ave-1c (PYPEQEQPI, SEQ ID NO: 12) in an IFN-γ ELISpot at 10 μg/mL. A) T cells responses from 19 CD patients following multi-grain 3-day challenge (9 non-responders not depicted). B) PBMC from 3 CD patients following 3-day barley challenge were tested against titrating concentrations of each peptide and additional barley peptide containing DQ2.5-hor-3b (PYPEQPQPY, SEQ ID NO: 5). Average Spot-forming units (SFU) from duplicate wells are shown.

FIG. 2 is a series of graphs showing T cells responses to homologous immuno-dominant avenin and barley peptides at the single cell level. T cell clones specific for DQ2.5-ave-1c (PYPEQEQPI, SEQ ID NO: 12) (Patient 2 TCC-01 and Patient 2 TCC-02), DQ2.5-hor-3a (PIPEQPQPY, SEQ ID NO: 4) (Patient 2 TCC-03 and Patient 6 TCC-01), or DQ2.5-hor-3b (PYPEQPQPY, SEQ ID NO: 5) (Patient 8 TCC-01) were tested by IFN-γ ELISpot against titrating concentrations of 3 barley and 3 avenin peptides containing homologous sequences. T cell cross-reactivity within and between grains was evident for all clones. Average Spot forming units (SFU) from duplicate wells are shown. *Assumed T cell epitope only.

FIG. 3 is a bar graph showing symptoms experienced by Celiac Disease patients following oral oats challenge. 89 CD patients undertook a 3-day challenge with three commercial varieties of oats. Patients kept symptom diaries and experienced a range of symptoms. Expressed as the percentage of patients that suffered symptoms, were asymptomatic, or did not complete the 3-day challenge.

FIG. 4 is a graph showing that T cell responses specific for barley peptides related to DQ2.5-hor-3a and DQ2.5-hor-3b can account for fresh polyclonal T cell responses to dominant avenin epitopes in CD patients. 7 HLA-DQ2.5+ CD patients undertook a 3-day challenge with either barley (B) or combined wheat, barley, and rye muffins (C). PBMC were tested for reactivity to peptides containing the epitopes DQ2.5-hor-3a (PIPEQPQPY, SEQ ID NO: 4), DQ2.5-ave-1c (PYPEQEQPI, SEQ ID NO: 12), or DQ2.5-hor-3b (PYPEQPQPY, SEQ ID NO: 5), or combinations of these peptides in an IFN-γ ELISpot at 25 μg/mL. Each patient is depicted by a separate symbol. Average Spot-forming units (SFU)/10⁶ PBMC calculated from duplicate wells are shown. The Friedman test was used to compare groups, with no statistical difference observed between the individual hordein peptides and the combinations of peptides.

FIG. 5 is a series of graphs showing the binding stability of avenin peptides and hordein peptides to a T cell clone.

### DETAILED DESCRIPTION

Oat prolamins (avenins) are tolerated by most celiac disease (CD) patients, but some patients have been shown to be sensitive to oats. As described herein, a study was undertaken to examine whether physico-chemical properties of avenins account for their reduced immunogenicity and to examine whether grain homology might provide an alternate explanation for the immunotoxicity of avenins in some CD patients. As described in Example 1, 89 CD patients undertook 3-day oral oats challenge and polyclonal avenin-specific T-cell responses were characterized. Grain cross-reactivity was tested using T cell clones. The study found that avenin-specific T cell responses were uncommon after oats ingestion. The most immunogenic was a partially deamidated avenin peptide (QYQPYPEQEQPILQQ, Genbank AAB32025 (7-21) [Q15 to E], SEQ ID NO: 39) that encompassed a possible homolog of an immuno-dominant epitope DQ2.5-hor-3a (PIPEQPQPY, SEQ ID NO: 4) from barley. Surprisingly, Genbank AAB32025 (7-21) [Q15 to E] or closely related sequences including the core 9 mer epitope PYPEQEQPI (SEQ ID NO: 12), also referred to herein as DQ2.5-ave-1c, or the related deamidated avenin sequences PYPEQEEPF (herein named: DQ2.5-ave-1a, SEQ ID NO: 40); and PYPEQEQPF (herein named: DQ2.5-ave-1b, SEQ ID NO: 41), were immunogenic in some patients after oral barley challenge. T cell clones isolated from HLA-DQ2.5+ CD patients specific for homologous oat and barley peptides were cross-reactive. Responses generated by cross-reactive high affinity hordein-specific T cells may overcome “below-threshold” responses usually observed to low level avenin epitope presentation in vivo and explain the toxicity of oats in CD.

Accordingly, aspects of the disclosure relate to compositions and methods for identifying and/or treating a subject sensitive to or likely to be sensitive to oats.

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984); J. Sambrook et al., Molecular Cloning: A Laboratory Manual, Cold Spring Harbour Laboratory Press (1989); T. A. Brown (editor), Essential Molecular Biology: A Practical Approach, Volumes 1 and 2, IRL Press (1991); D. M. Glover and B. D. Hames (editors), DNA Cloning: A Practical Approach, Volumes 1-4, IRL Press (1995 and 1996); F. M. Ausubel et al. (editors), Current Protocols in Molecular Biology, Greene Pub. Associates and Wiley-Interscience (1988, including all updates until present); Ed Harlow and David Lane (editors) Antibodies: A Laboratory Manual, Cold Spring Harbour Laboratory, (1988); and J. E. Coligan et al. (editors), Current Protocols in Immunology, John Wiley & Sons (including all updates until present).

The term “Celiac disease” refers to an immune-mediated systemic disorder elicited by gluten and related prolamins in genetically susceptible individuals, characterized by the presence of a variable combination of gluten-dependent clinical manifestations, celiac disease-specific antibodies, human leukocyte antigen (HLA)-DQ2 and HLA-DQ8 haplotypes, and enteropathy. The disease encompasses a spectrum of conditions characterised by an inappropriate CD4⁺ T cell response to gluten, or a peptide thereof. The severe form of celiac disease is characterised by a flat small intestinal mucosa (hyperplastic villous atrophy) and other forms are characterised by milder histological abnormalities in the small intestine, such as intra-epithelial lymphocytosis without villous atrophy. Serological abnormalities associated with celiac disease include the presence of autoantibodies specific for tissue transglutaminase-2, and antibodies specific for deamidated gluten-derived peptides. The clinical manifestations associated with celiac disease can include fatigue, chronic diarrhoea, malabsorption of nutrients, weight loss, abdominal distension, anaemia as well as a substantially enhanced risk for the development of osteoporosis and intestinal malignancies (lymphoma and carcinoma). A central feature in the current definitive diagnosis of celiac disease is that intestinal histology, celiac disease-specific serology and clinical abnormalities resolve or improve with exclusion of dietary gluten.

The term “subject” includes inter alia an individual, patient, target, host or recipient regardless of whether the subject is a human or non-human animal including mammalian species and also avian species. The term “subject”, therefore, includes a human, non-human primate (for example, gorilla, marmoset, African Green Monkey), livestock animal (for example, sheep, cow, pig, horse, donkey, goat), laboratory test animal (for example, rat, mouse, rabbit, guinea pig, hamster), companion animal (for example, dog, cat), captive wild animal (for example, fox, deer, game animals) and avian species including poultry birds (for example, chickens, ducks, geese, turkeys). The preferred subject, however, is a human. In some embodiments, the subject is a human on a gluten-free diet. In some embodiments, the subject is a human who is HLA-DQ2.5 positive. In some embodiments, the subject is a human who is HLA-DQ8 positive. In some embodiments, the subject is a human who is HLA-DQ2.5 positive and HLA-DQ8 negative. In some embodiments, the subject is human who is HLA-DQ2.5 positive and HLA-DQ8 positive.

### Methods of Identifying a Subject

In some aspects, the disclosure relates to methods for identifying a subject as sensitive to or likely sensitive to oats.

In some embodiments, the method comprises determining a T cell response to a barley peptide as described herein in a sample comprising a T cell from the subject and identifying the subject as (i) sensitive to or likely sensitive to oats if the T cell response to the barley peptide is elevated compared to a control T cell response, or (ii) not sensitive to or likely not sensitive to oats if the T cell response to the barley peptide is reduced compared to the control T cell response or the same as the control T cell response.

In some embodiments, the method comprises determining a T cell response to an oat peptide as described herein a sample comprising a T cell from the subject; and identifying the subject as (i) sensitive to or likely sensitive to oats if the T cell response to the oat peptide is elevated compared to a control T cell response, or (ii) not sensitive to or likely not sensitive to oats if the T cell response to the oat peptide is reduced compared to the control T cell response or the same as the control T cell response.

T cells responses and methods of measuring T cell responses are described herein. In some embodiments, the step of determining comprises contacting the sample with a composition comprising the barley peptide or oat peptide and measuring a T cell response to the barley peptide or oat peptide. Without wishing to be bound by theory, it is believed that the barley peptide or oat peptide serves as an active component causing the activation and/or mobilization of CD4+ T cells in a subject who has Celiac disease. Thus, in some embodiments, the T cell or T cell response referred to in any of the methods provided is a CD4+ T cell or CD4+ T cell response. In some embodiments, the subject has or is suspected of having Celiac disease.

In some embodiments, a method described herein further comprises performing a challenge as described herein.

In some embodiments, a method described herein further comprises performing an additional test, particularly if the subject is identified as sensitive to or likely sensitive to oats. In some embodiments, the additional test comprises measuring a T cell response to a second peptide. For example, if the first peptide is a barley peptide, the second peptide may be an oat peptide as described herein. In another example, if the first peptide is an oat peptide, the second peptide may be a barley peptide as described herein.

In some embodiments, a method described herein comprises a step of providing a treatment to a subject identified as being sensitive to or likely to be sensitive to oats. In some embodiments, a method described herein comprises a step of providing information to the subject about a treatment. In some embodiments, a method described herein comprises a step of recommending an oats-free diet, or providing information about such a diet, if the subject is identified as sensitive to or likely sensitive to oats. Information can be given orally or in written form, such as with written materials. Written materials may be in an electronic form. In some embodiments, treatment comprises administration of a composition as described herein, such as a vaccine composition.

### Peptides

The terms “peptide”, “polypeptide”, and “protein” can generally be used interchangeably and also encompass pharmaceutical salts thereof. However, the term “peptide” is typically used to refer to relatively short molecules comprising less than 50, more preferably less than 25, amino acids.

The overall length of each peptide defined herein may be, for example, 7 to 50 amino acids, such as 7, 8, 9 10, 11, 12, 13, 14, 15, 16, 17, 18, 19, 20, 21, 22, 23, 24, 25, 30, 35, 40, 45, or 50 amino acids, or any integer in between. It is contemplated that shorter peptides may prove useful, particularly those that are 20 or fewer amino acids in length, in therapeutics to reduce the likelihood of anaphylaxis but longer peptides with multiple epitopes are likely to be as effective as multiple short peptides, for example, in functional T cell-based diagnostics in vitro.

In some embodiments, the peptide is a barley peptide, such as a hordein peptide. Hordein is a prolamin glycoprotein found in barley. A “hordein peptide” is a peptide encompassing an amino acid sequence found within a hordein protein, including deamidated variants thereof containing one or more glutamine to glutamate substitutions. Conservative substitution of glutamate to aspartate is also contemplated (see, e.g., Anderson et al. Antagonists and non-toxic variants of the dominant wheat gliadin T-cell epitope in coeliac disease. Gut 2006; 55(4): 485-491). In some embodiments, a hordein peptide comprises an amino acid sequence of Genbank 1103203A (34-42) PIPQQPQPY (SEQ ID NO: 1), Genbank CAA60681.1 (35-43) PYPQQPQPY (SEQ ID NO: 2), or Genbank CAA37729.1 (28-36) PFPQQPQPY (SEQ ID NO: 3), or the partially deamidated homologs Genbank 1103203A (34-42) [Q37 to E] PIPEQPQPY (SEQ ID NO: 4), Genbank CAA60681.1 (35-43) [Q38 to E] PYPEQPQPY (SEQ ID NO: 5), or Genbank CAA37729.1 (28-36) [Q31 to E] PFPEQPQPY (SEQ ID NO: 6). In some embodiments, a hordein peptide comprises the amino acid sequence PIPQQPQPY (SEQ ID NO: 1), PYPQQPQPY (SEQ ID NO: 2), PFPQQPQPY (SEQ ID NO: 3), PIPEQPQPY (SEQ ID NO: 4), PYPEQPQPY (SEQ ID NO: 5), PFPEQPQPY (SEQ ID NO: 6), PIPDQPQPY (SEQ ID NO: 7), PYPDQPQPY (SEQ ID NO: 8), or PFPDQPQPY (SEQ ID NO: 9). In some embodiments, a hordein peptide comprises the amino acid sequence PIPQQPQPY (SEQ ID NO: 1), PIPEQPQPY (SEQ ID NO: 4) or PIPDQPQPY (SEQ ID NO: 7). In some embodiments, a hordein peptide comprises the amino acid sequence PIPEQPQPY (SEQ ID NO: 4).

In some embodiments, the peptide is an oat peptide, such as an avenin peptide. Avenin is a prolamin glycoprotein found in oats. An “avenin peptide” is a peptide encompassing an amino acid sequence found within an avenin protein, including deamidated variants thereof containing one or more glutamine to glutamate substitutions. Conservative substitution of glutamate to aspartate is also contemplated. In some embodiments, an avenin peptide comprises the amino acid sequence Genbank AAB32025 (10-18) PYPEQQQPI (SEQ ID NO: 11) or its partially deamidated homolog Genbank AAB32025 (10-18) [Q15 to E] PYPEQEQPI (SEQ ID NO: 12). In some embodiments, an avenin peptide comprises the amino acid sequence PYPEQQQPI (SEQ ID NO: 11), PYPEQEQPI (SEQ ID NO: 12), PYPEQDQPI (SEQ ID NO: 13), PYPDQEQPI (SEQ ID NO: 14) or PYPDQDQPI (SEQ ID NO: 15). In some embodiments, the avenin peptide comprises the amino acid sequence PYPEQEQPI (SEQ ID NO: 12). In some embodiments, the avenin peptide comprises the amino acid sequence Genbank AAB32025 (8-21) YQPYPEQQQPILQQ (SEQ ID NO: 16) or its partially deamidated homolog Genbank AAB32025 (8-21) [Q15 to E] YQPYPEQEQPILQQ (SEQ ID NO: 17). In some embodiments, the avenin peptide comprises the amino acid sequence YQPYPEQQQPILQQ (SEQ ID NO: 16), YQPYPEQEQPILQQ (SEQ ID NO: 17), YQPYPEQDQPILQQ (SEQ ID NO: 36), YQPYPDQEQPILQQ (SEQ ID NO: 37) or YQPYPDQDQPILQQ (SEQ ID NO: 38). In some embodiments, the avenin peptide comprises the amino acid sequence YQPYPEQEQPILQQ (SEQ ID NO: 17). In some embodiments, the avenin peptide comprises the amino acid sequence of Genbank AAA32714.1 (25-40) EQYQPYPEQQPFMQPL (SEQ ID NO: 18), Genbank AAB23365.1 (3-18) TVQYDPSEQYQPYPEQ (SEQ ID NO: 19), Genbank AAA32716.1 (20-39) TTTVQYNPSEQYQPYPEQQE (SEQ ID NO: 20) or their partially deamidated homologs Genbank AAA32714.1 (25-40) [Q32 to E] EQYQPYPEEQPFMQPL (SEQ ID NO: 21), Genbank AAA32716.1 (20-39)[Q38 to E] TTTVQYNPSEQYQPYPEQEE (SEQ ID NO: 22), Genbank AAB23365.1 (9-24) SEQYQPYPEQQQPFVQ (SEQ ID NO: 23), Genbank Q09097.1 (1-20) TTTVQYDPSEQYQPYPEQQE (SEQ ID NO: 24), Genbank AAB23365.1 (9-24) [Q19 to E] SEQYQPYPEQEQPFVQ (SEQ ID NO: 25), Genbank AAB32025 (7-22) [Q15 to E] QYQPYPEQEQPILQQQ (SEQ ID NO: 27), Genbank AAB32025 (7-22) QYQPYPEQQQPILQQQ (SEQ ID NO: 26), Genbank AAA32715.1 (19-38) AQFDPSEQYQPYPEQQQPIL (SEQ ID NO: 28), Genbank AAB23365.1 (10-29) EQYQPYPEQQQPFVQQQPPF (SEQ ID NO: 29), Genbank Q09097.1 (9-28) [Q19 to E] SEQYQPYPEQEEPFVQQQPP (SEQ ID NO: 32), Genbank P14812.1 (402-421) NNHGQTVFNDILRRGQLLII (SEQ ID NO: 30), Genbank Q09095.1 (2-21) [Q12 and Q18 to E] SEQYQPYPEQEQPFLQEQPL (SEQ ID NO: 33), Genbank AAB23365.1 (10-29) [Q19 to E] EQYQPYPEQEQPFVQQQPPF (SEQ ID NO: 34), Genbank AAB32025 (4-23) [Q15, Q22, and Q23 to E] PSEQYQPYPEQEQPILQQEE (SEQ ID NO: 35), or Genbank Q09095.1 (3-22) EQYQPYPEQQQPFLQQQPLE (SEQ ID NO: 31).

In some embodiments, one or more glutamate residues of a peptide may be generated by tissue transglutaminase (tTG) deamidation activity upon one or more glutamine residues of the peptide. This deamidation of glutamine to glutamate causes the generation of peptides that can bind to HLA-DQ2 or -DQ8 molecules with high affinity. This reaction may occur in vitro by contacting the peptide composition with tTG outside of the subject (e.g., prior to or during contact of a peptide composition with a sample comprising T cells from a subject) or in vivo following administration through deamidation via tTG in the body. Deamidation of a peptide may also be accomplished by synthesizing a peptide de novo with glutamate residues in place of one or more glutamine residues, and thus deamidation does not necessarily require use of tTG.

In some embodiments, a peptide is modified during or after translation or synthesis (for example, by farnesylation, prenylation, myristoylation, glycosylation, palmitoylation, acetylation, phosphorylation [such as phosphotyrosine, phosphoserine or phosphothreonine], amidation, derivatisation by known protecting/blocking groups, proteolytic cleavage, linkage to an antibody molecule or other cellular ligand, and the like). Any of the numerous chemical modification methods known within the art may be utilised including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

The phrases “protecting group” and “blocking group” as used herein, refers to modifications to the peptide, which protect it from undesirable chemical reactions, particularly in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/or increased half-life will be known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, amide, carboxy, hydroxy or halogen (for example, fluorine) group, or a carbohydrate group. Typically, the modification is present on the N- and/or C-terminus. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell. The peptide may also be chemically modified at the level of amino acid side chains, of amino acid chirality, and/or of the peptide backbone.

Particular changes can be made to the peptides to improve resistance to degradation or optimise solubility properties or otherwise improve bioavailability compared to the parent peptide, thereby providing peptides having similar or improved therapeutic, diagnostic and/or pharmacokinetic properties. A preferred such modification includes the use of an N-terminal pyroglutamate and/or a C-terminal amide. Such modifications have been shown previously to significantly increase the half-life and bioavailability of the peptides compared to the parent peptides having a free N- and C-terminus.

In a particular embodiment, a composition comprising one or more peptides of any of the peptides described herein is contemplated. Compositions are further described herein.

Certain peptides described herein may exist in particular geometric or stereoisomeric forms. The present disclosure contemplates all such forms, including cis-(Z) and trans-(E) isomers, R- and S-enantiomers, diastereomers, (D)-isomers, (L)-isomers, the racemic mixtures thereof, and other mixtures thereof, as, falling within the scope of the disclosure. Additional asymmetric carbon atoms may be present in a substituent, such as an alkyl group. All such isomers, as well as mixtures thereof, are intended to be included in this disclosure. In another example, to prevent cleavage by peptidases, any one or more of the peptides may include a non-cleavable peptide bond in place of a particularly sensitive peptide bond to provide a more stable peptide. Such non cleavable peptide bonds may include beta amino acids.

In certain embodiments, any one or more of the peptides may include a functional group, for example, in place of the scissile peptide bond, which facilitates inhibition of a serine-, cysteine- or aspartate-type protease, as appropriate. For example, the disclosure includes a peptidyl diketone or a peptidyl keto ester, a peptide haloalkylketone, a peptide sulfonyl fluoride, a peptidyl boronate, a peptide epoxide, a peptidyl diazomethane, a peptidyl phosphonate, isocoumarins, benzoxazin-4-ones, carbamates, isocyantes, isatoic anhydrides or the like. Such functional groups have been provided in other peptide molecules, and general routes for their synthesis are known.

The peptides may be in a salt form, preferably, a pharmaceutically acceptable salt form. “A pharmaceutically acceptable salt form” includes the conventional non-toxic salts or quaternary ammonium salts of a peptide, for example, from non-toxic organic or inorganic acids. Conventional non-toxic salts include, for example, those derived from inorganic acids such as hydrochloride, hydrobromic, sulphuric, sulfonic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

### Peptide Production

The peptides can be prepared in any suitable manner. For example, the peptides can be recombinantly and/or synthetically produced.

The peptides may be synthesised by standard chemistry techniques, including synthesis by an automated procedure using a commercially available peptide synthesiser. In general, peptides may be prepared by solid-phase peptide synthesis methodologies which may involve coupling each protected amino acid residue to a resin support, preferably a 4-methylbenzhydrylamine resin, by activation with dicyclohexylcarbodiimide to yield a peptide with a C-terminal amide. Alternatively, a chloromethyl resin (Merrifield resin) may be used to yield a peptide with a free carboxylic acid at the C-terminal. After the last residue has been attached, the protected peptide-resin is treated with hydrogen fluoride to cleave the peptide from the resin, as well as deprotect the side chain functional groups. Crude product can be further purified by gel filtration, high pressure liquid chromatography (HPLC), partition chromatography, or ion-exchange chromatography.

If desired, and as outlined above, various groups may be introduced into the peptide of the composition during synthesis or during expression, which allow for linking to other molecules or to a surface. For example, cysteines can be used to make thioethers, histidines for linking to a metal ion complex, carboxyl groups for forming amides or esters, amino groups for forming amides, and the like.

The peptides may also be produced using cell-free translation systems. Standard translation systems, such as reticulocyte lysates and wheat germ extracts, use RNA as a template; whereas “coupled” and “linked” systems start with DNA templates, which are transcribed into RNA then translated.

Alternatively, the peptides may be produced by transfecting host cells with expression vectors that comprise a polynucleotide(s) that encodes one or more peptides.

For recombinant production, a recombinant construct comprising a sequence which encodes one or more of the peptides is introduced into host cells by conventional methods such as calcium phosphate transfection, DEAE-dextran mediated transfection, microinjection, cationic lipid-mediated transfection, electroporation, transduction, scrape lading, ballistic introduction or infection.

One or more of the peptides may be expressed in suitable host cells, such as, for example, mammalian cells (for example, COS, CHO, BHK, 293 HEK, VERO, HeLa, HepG2, MDCK, W138, or NIH 3T3 cells), yeast (for example, Saccharomyces or Pichia), bacteria (for example, E. coli, P. pastoris, or B. subtilis), insect cells (for example, baculovirus in Sf9 cells) or other cells under the control of appropriate promoters using conventional techniques. Following transformation of the suitable host strain and growth of the host strain to an appropriate cell density, the cells are harvested by centrifugation, disrupted by physical or chemical means, and the resulting crude extract retained for further purification of the peptide or variant thereof.

Suitable expression vectors include, for example, chromosomal, non-chromosomal and synthetic polynucleotides, for example, derivatives of SV40, bacterial plasmids, phage DNAs, yeast plasmids, vectors derived from combinations of plasmids and phage DNAs, viral DNA such as vaccinia viruses, adenovirus, adeno-associated virus, lentivirus, canary pox virus, fowl pox virus, pseudorabies, baculovirus, herpes virus and retrovirus. The polynucleotide may be introduced into the expression vector by conventional procedures known in the art.

The polynucleotide which encodes one or more peptides may be operatively linked to an expression control sequence, i.e., a promoter, which directs mRNA synthesis. Representative examples of such promoters include the LTR or SV40 promoter, the E. coli lac or trp, the phage lambda PL promoter and other promoters known to control expression of genes in prokaryotic or eukaryotic cells or in viruses. The expression vector may also contain a ribosome binding site for translation initiation and a transcription terminator. The expression vectors may also include an origin of replication and a selectable marker, such as the ampicillin resistance gene of E. coli to permit selection of transformed cells, i.e., cells that are expressing the heterologous polynucleotide. The nucleic acid molecule encoding one or more of the peptides may be incorporated into the vector in frame with translation initiation and termination sequences.

One or more of the peptides can be recovered and purified from recombinant cell cultures (i.e., from the cells or culture medium) by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography, lectin chromatography, and HPLC. Well known techniques for refolding proteins may be employed to regenerate active conformation when the peptide is denatured during isolation and or purification.

To produce a glycosylated peptide, it is preferred that recombinant techniques be used. To produce a glycosylated peptide, it is preferred that mammalian cells such as, COS-7 and Hep-G2 cells be employed in the recombinant techniques.

The peptides can also be prepared by cleavage of longer peptides, especially from food extracts.

Pharmaceutically acceptable salts of the peptides can be synthesised from the peptides which contain a basic or acid moiety by conventional chemical methods. Generally, the salts are prepared by reacting the free base or acid with stoichiometric amounts or with an excess of the desired salt-forming inorganic or organic acid or base in a suitable solvent.

### T Cell Responses and Measurement Thereof

Aspects of the disclosure relate to a determination or measurement of a T cell response in a sample comprising T cells from a subject. In some embodiments, a composition comprising oats or barley, or a peptide thereof, is administered to a subject and, preferably, is capable of activating a CD4⁺ T cell in a subject, e.g., a subject with Celiac disease. In some embodiments, a composition comprising an oat avenin or a barley hordein, or a peptide thereof, is administered to a subject and, preferably, is capable of activating a CD4+ T cell in a subject. The term “activate” or “activating” or “activation” in relation to a CD4⁺ T cell refers to the presentation by an MHC molecule of an epitope on one cell to an appropriate T cell receptor on a second CD4⁺ T cell, together with binding of a co-stimulatory molecule by the CD4⁺ T cell, thereby eliciting a “T cell response”, in this example a CD4⁺ T cell response. Such a T cell response can be measured ex vivo, e.g., by measuring a T cell response in a sample comprising T cells from the subject.

As described herein, an elevated T cell response, such as an elevated CD4⁺ T cell response, from a sample comprising T cells from a subject after administration of a composition compared to a control T cell response can correlate with the presence or absence of Celiac disease in the subject. Accordingly, aspects of the disclosure relate to methods that comprise determining or measuring a T cell response in a sample comprising T cells from a subject, e.g., having or suspected of having Celiac disease.

In some embodiments, measuring a T cell response in a sample comprising T cells from a subject comprises contacting the sample with a composition comprising a peptide as described herein. For example, whole blood or PBMCs obtained from a subject who has been exposed to an oat peptide or a barley peptide (e.g., by a challenge as described herein) may be contacted with the composition in order to stimulate T cells in the whole blood sample.

Measuring a T cell response can be accomplished using any assay known in the art (see, e.g., Molecular Cloning: A Laboratory Manual, J. Sambrook, et al., eds., Third Edition, Cold Spring Harbor Laboratory Press, Cold Spring Harbor, N.Y., 2001, Current Protocols in Molecular Biology, F. M. Ausubel, et al., eds., John Wiley & Sons, Inc., New York. Microarray technology is described in Microarray Methods and Protocols, R. Matson, CRC Press, 2009, or Current Protocols in Molecular Biology, F. M. Ausubel, et al., eds., John Wiley & Sons, Inc., New York). In some embodiments, measuring a T cell response comprises an MHC Class II tetramer assay, such as flow cytometry with MHC Class II tetramer staining (see, e.g., Raki M, Fallang L E, Brottveit M, Bergseng E, Quarsten H, Lundin K E, Sollid L M: Tetramer visualization of gut-homing gluten-specific T cells in the peripheral blood of Celiac disease patients. Proceedings of the National Academy of Sciences of the United States of America 2007; Anderson R P, van Heel D A, Tye-Din J A, Barnardo M, Salio M, Jewell D P, Hill A V: T cells in peripheral blood after gluten challenge in coeliac disease. Gut 2005, 54(9):1217-1223; Brottveit M, Raki M, Bergseng E, Fallang L E, Simonsen B, Lovik A, Larsen S, Loberg E M, Jahnsen F L, Sollid L M et al: Assessing possible Celiac disease by an HLA-DQ2-gliadin Tetramer Test. The American journal of gastroenterology 2011, 106(7):1318-1324; and Anderson R P, Degano P, Godkin A J, Jewell D P, Hill A V: In vivo antigen challenge in Celiac disease identifies a single transglutaminase-modified peptide as the dominant A-gliadin T cell epitope. Nature Medicine 2000, 6(3):337-342).

In some embodiments, measuring a T cell response in a sample comprising T cells from a subject comprises measuring a level of at least one cytokine in the sample. In some embodiments, measuring a T cell response in a sample comprising T cells from a subject comprises contacting the sample with a composition comprising a peptide as described herein and measuring a level of at least one cytokine in the sample. In some embodiments, the at least one cytokine is at least one pro-inflammatory cytokine such as IL-2, IFN-γ, IL-4, IL-5, IP-10, IL-13, and IL-17, or chemokines such as MCP-1 and GM-CSF released, e.g., by monocytes or granulocytes, as a result of secretion of these cytokines. In some embodiments, the at least one cytokine is IFN-γ or IP-10. In some embodiments, the at least one cytokine is IP-10. In some embodiments, the at least one cytokine is IFN-γ.

Interferon-γ (IFN-γ, also called IFNG, IFG, and IFI) is a dimerized soluble cytokine of the type II class of interferons. IFN-γ typically binds to a heterodimeric receptor consisting of Interferon γ receptor 1 (IFNGR1) and Interferon γ receptor 2 (IFNGR2). IFN-γ can also bind to the glycosaminoglycan heparan sulfate (HS). IFN-γ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops in a subject. In humans, the IFN-γ protein is encoded by the IFNG gene. The Genbank number for the human IFNG gene is 3458. Exemplary Genbank mRNA transcript IDs and protein IDs for IFN-γ are NM_000619.2 and NP_000610.2, respectively.

IFN-γ inducible protein-10 (IP-10, also referred to as C-X-C motif chemokine 10, CXCL10, small-inducible cytokine B10, SCYB10, C7, IFI10, crg-2, gIP-10, or mob-1) is a protein that in humans is encoded by the CXCL10 gene. IP-10 is a small cytokine belonging to the CXC chemokine family and binds to the chemokine receptor CXCR3. The Genbank ID number for the human CXCL10 gene is 3627. Exemplary Genbank mRNA transcript IDs and protein IDs for IP-10 are NM_001565.3 and NP_001556.2, respectively.

In some embodiments, measuring a T cell response comprises measuring a level of at least one cytokine. Levels of at least one cytokine include levels of cytokine RNA, e.g., mRNA, and/or levels of cytokine protein. In a preferred embodiment, levels of the at least one cytokine are protein levels.

Assays for detecting cytokine RNA include, but are not limited to, Northern blot analysis, RT-PCR, sequencing technology, RNA in situ hybridization (using e.g., DNA or RNA probes to hybridize RNA molecules present in the sample), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface (e.g., a glass wafer with addressable location, such as Affymetrix microarray (Affymetrix®, Santa Clara, Calif.)). Designing nucleic acid binding partners, such as probes, is well known in the art. In some embodiments, the nucleic acid binding partners bind to a part of or an entire nucleic acid sequence of at least one cytokine, e.g., IFN-γ, the sequence(s) being identifiable using the Genbank IDs described herein.

Assays for detecting protein levels include, but are not limited to, immunoassays (also referred to herein as immune-based or immuno-based assays, e.g., Western blot, ELISA, and ELISpot assays), Mass spectrometry, and multiplex bead-based assays. Binding partners for protein detection can be designed using methods known in the art and as described herein. In some embodiments, the protein binding partners, e.g., antibodies, bind to a part of or an entire amino acid sequence of at least one cytokine, e.g., IFN-γ, the sequence(s) being identifiable using the Genbank IDs described herein. Other examples of protein detection and quantitation methods include multiplexed immunoassays as described for example in U.S. Pat. Nos. 6,939,720 and 8,148,171, and published U.S. Patent Application No. 2008/0255766, and protein microarrays as described for example in published U.S. Patent Application No. 2009/0088329.

In a preferred embodiment, measuring a level of at least one cytokine comprises an enzyme-linked immunosorbent assay (ELISA) or enzyme-linked immunosorbent spot (ELISpot) assay. ELISA and ELISpot assays are well known in the art (see, e.g., U.S. Pat. Nos. 5,939,281, 6,410,252, and 7,575,870; Czerkinsky C, Nilsson L, Nygren H, Ouchterlony O, Tarkowski A (1983) “A solid-phase enzyme-linked immunospot (ELISPOT) assay for enumeration of specific antibody-secreting cells”. J Immunol Methods 65 (1-2): 109-121 and Lequin R (2005). “Enzyme immunoassay (EIA)/enzyme-linked immunosorbent assay (ELISA)”. Clin. Chem. 51 (12): 2415-8).

An exemplary ELISA involves at least one binding partner, e.g., an antibody or antigen-binding fragment thereof, with specificity for the at least one cytokine, e.g., IFN-γ. The sample with an unknown amount of the at least one cytokine can be immobilized on a solid support (e.g., a polystyrene microtiter plate) either non-specifically (via adsorption to the surface) or specifically (via capture by another binding partner specific to the same at least one cytokine, as in a “sandwich” ELISA). After the antigen is immobilized, the binding partner for the at least one cytokine is added, forming a complex with the immobilized at least one cytokine. The binding partner can be attached to a detectable label as described herein (e.g., a fluorophor or an enzyme), or can itself be detected by an agent that recognizes the at least one cytokine binding partner that is attached to a detectable label as described herein (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

An exemplary ELISpot assay involves a binding agent for the at least one cytokine (e.g., an anti-IFN-γ) that is coated aseptically onto a PVDF (polyvinylidene fluoride)-backed microplate. Cells of interest (e.g., peripheral blood mononuclear cells) are plated out at varying densities, along with antigen (e.g., a peptide as described herein), and allowed to incubate for a period of time (e.g., about 24 hours). The at least one cytokine secreted by activated cells is captured locally by the binding partner for the at least one cytokine on the high surface area PVDF membrane. After the at least one cytokine is immobilized, a second binding partner for the at least one cytokine is added, forming a complex with the immobilized at least one cytokine. The binding partner can be linked to a detectable label (e.g., a fluorophor or an enzyme), or can itself be detected by an agent that recognizes the binding partner for the at least one cytokine (e.g., a secondary antibody) that is linked to a detectable label (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

In some embodiments, a level of at least one cytokine is measured using an ELISA. As an exemplary method, at least one peptide as defined herein is dried onto the inner wall of a blood collection tube. A negative control tube containing no antigen is provided. A positive control tube containing a mitogen is also provided. Blood from a subject is drawn into each of the three tubes. Each tube is agitated to ensure mixing. The tubes are then incubated at 37 degrees Celsius, preferably immediately after blood draw or at least within about 16 hours of collection. After incubation, the cells are separated from the plasma by centrifugation. The plasma is then loaded into an ELISA plate for detection of levels of at least one cytokine (e.g., IFN-γ) present in the plasma. A standard ELISA assay as described above can then be used to detect the levels of the at least one cytokine present in each plasma sample. In some embodiments, a T cell response measurement in a sample obtained from the subject after a challenge as described herein is detected using any of the methods above or any other appropriate method and is then compared to a control T cell response, e.g., a T cell response measurement in a sample obtained before challenge or a T cell response measurement in a sample from a control subject or subjects. Exemplary control T cell responses include, but are not limited to, a T cell response in a sample obtained from a diseased subject(s) (e.g., subject(s) with Celiac disease), a healthy subject(s) (e.g., subject(s) without Celiac disease) or a T cell response in a sample obtained from a subject before or during a challenge as described herein. In some embodiments, a control T cell response is measured using any of the methods above or any other appropriate methods. In some embodiments, the same method is used to measure T cell response in the sample of the subject and the control sample.

In some embodiments, a T cell response is compared to a control T cell response. In some embodiments, if the control T cell response is a T cell response in a sample from a healthy control subject or subjects, then an elevated T cell response compared to the control T cell response is indicative that the subject is sensitive to or likely sensitive to oats while a reduced or equal T cell response compared to the control T cell response is indicative that the subject is not sensitive to or likely not sensitive to oats. In some embodiments, if the control T cell response is a T cell response in a sample from the subject before a challenge as described herein, then an elevated T cell response compared to the control T cell response is indicative that the subject is sensitive to or likely sensitive to oats while a reduced or equal T cell response compared to the control T cell response is indicative that the subject is not sensitive to or likely not sensitive to oats. In some embodiments, if the control T cell response is a T cell response in a sample from a diseased control subject or subjects, then an elevated or equal T cell response compared to the control T cell response is indicative that the subject is sensitive to or likely sensitive to oats while a reduced T cell response compared to the control T cell response is indicative that the subject is not sensitive to or likely not sensitive to oats.

An elevated T cell response includes a response that is, for example, 1%, 5%, 10%, 20%, 30%, 40%, 50%, 60%, 70%, 80%, 90%, 100%, 150%, 200%, 300%, 400%, 500% or more above a control T cell response. A reduced T cell response includes a response that is, for example, 1%, 5%, 10%, 20%, 30%, 40%, 50%, 60%, 70%, 80%, 90%, 100%, 150%, 200%, 300%, 400%, 500% or more below a control T cell response. In some embodiments, a second control T cell response is contemplated. In some embodiments, the second control T cell response is a negative control T cell response. Exemplary negative controls include, but are not limited to, a T cell response in a sample that has been contacted with a non-T cell-activating peptide (e.g., a peptide not recognized by T cells present in a sample from a subject), such as a non-CD4⁺-T cell-activating peptide, or a T cell response in sample that has not been contacted with a T cell-activating peptide (e.g., contacting the sample with a saline solution containing no peptides), such as a CD4⁺ T cell-activating peptide. Such a second control T cell response can be measured using any of the methods above or any other appropriate methods. In some embodiments, the second control T cell response is a positive control T cell response. Exemplary positive controls include, but are not limited to, a T cell response in a sample that has been contacted with a mitogen (e.g., phytohaemagglutinin, concanavalin A, lipopolysaccharide, or pokeweed mitogen). Positive and/or negative controls may be used to determine that an assay, such as an ELISA or ELISpot assay, is not defective or contaminated.

### Challenge

In some embodiments, methods provided herein comprise a challenge or a sample obtained from a subject before, during, or after a challenge.

Generally, a challenge comprises administering to the subject a composition comprising oats or barley, or a peptide thereof (e.g., a composition comprising an oat avenin or a barley hordein, or a peptide thereof), in some form for a defined period of time in order to activate the immune system of the subject, e.g., through activation of barley- and/or oats-reactive T cells and/or mobilization of such T cells in the subject. Methods of challenges, e.g., gluten challenges, are well known in the art and include oral, submucosal, supramucosal, and rectal administration of peptides or proteins (see, e.g., Can J Gastroenterol. 2001. 15(4):243-7. In vivo gluten challenge in celiac disease. Ellis H J, Ciclitira P J; Mol Diagn Ther. 2008. 12(5):289-98. Celiac disease: risk assessment, diagnosis, and monitoring. Setty M, Hormaza L, Guandalini S; Gastroenterology. 2009; 137(6):1912-33. Celiac disease: from pathogenesis to novel therapies. Schuppan D, Junker Y, Barisani D; J Dent Res. 2008; 87(12):1100-1107. Orally based diagnosis of celiac disease: current perspectives. Pastore L, Campisi G, Compilato D, and Lo Muzio L; Gastroenterology. 2001; 120:636-651. Current Approaches to Diagnosis and Treatment of Celiac Disease: An Evolving Spectrum. Fasano A and Catassi C; Clin Exp Immunol. 2000; 120:38-45. Local challenge of oral mucosa with gliadin in patients with coeliac disease. Lahteenoja M, Maki M, Viander M, Toivanen A, Syrjanen S; Clin Exp Immunol. 2000; 120:10-11. The mouth-an accessible region for gluten challenge. Ellis H and Ciclitira P; Clinical Science. 2001; 101:199-207. Diagnosing coeliac disease by rectal gluten challenge: a prospective study based on immunopathology, computerized image analysis and logistic regression analysis. Ensari A, Marsh M, Morgan S, Lobley R, Unsworth D, Kounali D, Crowe P, Paisley J, Moriarty K, and Lowry J; Gut. 2005; 54:1217-1223. T cells in peripheral blood after gluten challenge in coeliac disease. Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). Traditionally, a challenge lasts for several weeks (e.g., 4 weeks or more) and involves high doses of orally administered peptides or proteins (usually in the form of baked foodstuff that includes the peptides or proteins). Some studies suggest that a shorter challenge, e.g., through use of as little as 3 days of oral challenge, is sufficient to activate and/or mobilize reactive T-cells (Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). Any such methods of challenge that are capable of activating the immune system of the subject, e.g., by activating oats- or barley-reactive T-cells and and/or mobilizing such T cells into blood are contemplated herein. An exemplary challenge is shown in Example 1.

In some embodiments, the challenge comprises administering a composition comprising barley and/or oats, or a peptide thereof, to the subject prior to determining a T cell response as described herein. The composition may further comprise, e.g., a wheat and/or rye, or a peptide thereof. In some embodiments, the challenge comprises administering a composition comprising a barley hordein and/or an oat avenin, or a peptide thereof, to the subject prior to determining a T cell response as described herein. The composition may further comprise, e.g., a wheat gliadin and/or a rye secalin, or a peptide thereof.

In some embodiments, the composition is administered to the subject more than once prior to determining the T cell response, and a sample is obtained from the subject after administration of the composition. In some embodiments, administration is daily for 3 days. In some embodiments, the sample is obtained from the subject 6 days after administration of the composition. In some embodiments, the subject has been on a gluten-free diet for at least 4 weeks prior to commencing the challenge.

In some embodiments, administration is oral. Suitable forms of oral administration include foodstuffs (e.g., baked goods such as breads, cookies, cakes, etc.), tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions or foodstuffs and such compositions may contain one or more agents including, for example, sweetening agents, flavoring agents, coloring agents and preserving agents in order to provide pharmaceutically elegant and palatable preparations.

In some embodiments, a challenge comprises administration of 100 g dry weight oats to the subject. In some embodiments, the oats contain less than 20 ppm wheat gluten contamination as measured by R5 ELISA. In some embodiments, the oats are commercially available oats selected from Uncle Toby's oats, Tilquhilie Pudding oats, or Freedom Food oats. In some embodiments, the oats are administered to the subject daily.

In some embodiments, a challenge comprises administration of 150 g dry weight pearl barley. In some embodiments, the pearl barley is cooked into a risotto. In some embodiments, the barley is commercially available barley from Ward McKenzie. In some embodiments, the barley is administered to the subject daily.

In some embodiments, a challenge comprises administration of 22-25 g dry weight of each of wheat, barley, and rye to a subject. In some embodiments, the wheat is wheat flour, barley flour, and rye flour. In some embodiments, the wheat, barley, and rye are baked into muffins. In some embodiments, the wheat is commercially available wheat from White Wings, the barley is commercially available barley from Four Leaf Milling, and the rye is commercially available rye from Four Leaf Milling.

In some embodiments, a sample is obtained from a subject before, during, and/or after a challenge as described herein. In some embodiments, the sample is a sample comprising a T cell. In some embodiments, the sample is contacted with a peptide as described herein, e.g., a oat or barley peptide. In some embodiments, a T cell response in the sample is measured as described herein.

In some embodiments, a challenge as described herein comprises a step of providing a treatment to a subject identified as being sensitive to or likely to be sensitive to oats. In some embodiments, a method described herein comprises a step of providing information to the subject about a treatment. In some embodiments, a method described herein comprises a step of recommending an oats-free diet, or providing information about such a diet, if the subject is identified as sensitive to or likely sensitive to oats. Information can be given orally or in written form, such as with written materials. Written materials may be in an electronic form. In some embodiments, treatment comprises administration of a composition as described herein, such as a vaccine composition.

### Samples

Samples, as used herein, refer to biological samples taken or derived from a subject, e.g., a subject having or suspected of having Celiac disease. Examples of samples include tissue samples or fluid samples. Examples of fluid samples are whole blood, plasma, serum, and other bodily fluids that comprise T cells. In some embodiments, the sample comprises T cells. In some embodiments, the sample comprises T cells and monocytes and/or granulocytes. In some embodiments, the sample comprising T cells comprise whole blood or peripheral blood mononuclear cells (PBMCs). The T cell may be a CD4+ T cell, e.g., an avenin- or hordein-reactive CD4+ T cell. In some embodiments, the methods described herein comprise obtaining or providing the sample. In some embodiments, the sample is obtained from a subject after a challenge as described herein. In some embodiments, a first and second sample are contemplated. Additional samples, e.g., third, fourth, fifth, etc., are also contemplated if additional measurements of a T cell response are desired. Such additional samples may be obtained from the subject at any time, e.g., before or after a challenge as described herein (e.g., before or after administration of a composition comprising a barely hordein or an oat avenin, or a peptide thereof).

### Controls and Control Subjects

In some embodiments, methods provided herein comprise measuring a control T cell response. In some embodiments, the control T cell response is a T cell response in a sample from the subject before or during a challenge as described herein.

In some embodiments, the control T cell response is a T cell response in a sample obtained from a control subject (or subjects). In some embodiments, a control subject has one or more HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein but does not have Celiac disease. In some embodiments, a control subject does not have any of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein. In some embodiments, a control subject is a healthy individual not having or suspected of having Celiac disease. In some embodiments, a control T cell response is a pre-determined value from a control subject or subjects, such that the control T cell response need not be measured every time the methods described herein are performed.

Embodiments for the comparison of T cell responses to control T cell responses are described elsewhere herein.

### Additional Testing

In some embodiments, methods described herein comprise additional testing of a subject (e.g., based on the results of the methods described herein). As used herein, “additional testing” describes use of at least one additional diagnostic method in addition to the methods provided herein or use of methods described herein in combination (e.g., measuring a T cell response to a barley peptide as described herein, followed by measuring a T cell response to an oats peptide as described herein). Any diagnostic method or combinations thereof for Celiac disease is contemplated as additional testing. Exemplary additional testing includes, but is not limited to, intestinal biopsy, serology (measuring the levels of one or more antibodies present in the serum), and genotyping (see, e.g., Walker-Smith J A, et al. Arch Dis Child 1990). Such additional testing may be performed as part of the methods described herein or after the methods described herein (e.g., as a companion diagnostic), or before use of the methods described herein (e.g., as a first-pass screen to eliminate certain subjects before use of the methods described herein, e.g., eliminating those that do not have one or more HLA-DQA and HLA-DQB susceptibility alleles).

When performing intestinal biopsies, generally multiple biopsies are taken from the first, second, and/or third part of the duodenum. Endoscopy has become the most convenient method of obtaining biopsies of the small-intestinal mucosa, but the older method of suction biopsy (with a Crosby capsule) can provide adequate samples. Left untreated, celiac disease (CD) affects the mucosa of the proximal small intestine, with damage gradually decreasing in severity towards the distal small intestine, although in severe cases the lesions can extend to the ileum. The degree of proximal damage varies greatly depending on the severity of the disease. The proximal damage may be very mild in “silent” cases, with only intra-epithelial lymphocytosis, and in potential celiac disease the mucosa is normal but persistent serological abnormalities in genetically susceptible individuals predicts eventual manifestation of histological abnormalities in the small intestine typical of celiac disease. Abnormalities in the gastric and rectal mucosa may be observed in some cases. Occasionally, the lesion in the duodenum/upper jejunum can be patchy, which may justify a second biopsy immediately in selected patients with positive endomysial antibody (EMA). However, this is only warranted if all three samples of the first biopsy show a normal histology. Histological abnormalities in the intestine of patients with celiac disease are responsive to gluten exclusion except in rare cases, usually older patients who have longstanding untreated celiac disease, that have so-called refractory celiac disease which may be complicated by intestinal lymphoma.

Detection of serum antibodies (serology) is also contemplated. The presence of such serum antibodies can be detected using methods known to those of skill in the art, e.g., by ELISA, histology, cytology, immunofluorescence or western blotting. Such antibodies include, but are not limited to: IgA ant-endomysial antibody (IgA EMA), IgA anti-tissue transglutaminase antibody (IgA tTG), IgA anti-deamidated gliadin peptide antibody (IgA DGP), and IgG anti-deamidated gliadin peptide antibody (IgG DGP).

IgA EMA: IgA endomysial antibodies bind to endomysium, the connective tissue around smooth muscle, producing a characteristic staining pattern that is visualized by indirect immunofluorescence. The target antigen has been identified as tissue transglutaminase (tTG or transglutaminase 2). IgA endomysial antibody testing is thought to be moderately sensitive and highly specific for untreated (active) Celiac disease.

IgA tTG: The antigen is tTG. Anti-tTG antibodies are thought to be highly sensitive and specific for the diagnosis of Celiac disease. Enzyme-linked immunosorbent assay (ELISA) tests for IgA anti-tTG antibodies are now widely available and are easier to perform, less observer-dependent, and less costly than the immunofluorescence assay used to detect IgA endomysial antibodies. The diagnostic accuracy of IgA anti-tTG immunoassays has been improved further by the use of human tTG in place of the nonhuman tTG preparations used in earlier immunoassay kits. Kits for IgA tTG are commercially available (INV 708760, 704525, and 704520, INOVA Diagnostics, San Diego, Calif.).

Deamidated gliadin peptide-IgA (DGP-IgA) and deamidated gliadin peptide-IgG (DGP-IgG) are also contemplated herein and can be evaluated with commercial kits (INV 708760, 704525, and 704520, INOVA Diagnostics, San Diego, Calif.).

Genetic testing (genotyping) is also contemplated. Subjects can be tested for the presence of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). Exemplary sequences that encode the DQA and DQB susceptibility alleles include HLA-DQA1*0501 (Genbank accession number: AF515813.1) HLA-DQA1*0505 (AH013295.2), HLA-DQB1*0201 (AY375842.1) or HLA-DQB1*0202 (AY375844.1). Methods of genetic testing are well known in the art (see, e.g., Bunce M, et al. Phototyping: comprehensive DNA typing for HLA-A, B, C, DRB1, DRB3, DRB4, DRBS & DQB1 by PCR with 144 primer mixes utilizing sequence-specific primers (PCR-SSP). Tissue Antigens 46, 355-367 (1995); Olerup O, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256.; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3: e2270). Subjects that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D, Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

### Compositions, Vaccine Compositions, and Administration

### Compositions and Vaccine Compositions

The disclosure also provides a composition comprising a peptide as described herein. In some embodiments, the peptide comprises the amino acid sequence PYPEQEQPI (SEQ ID NO: 12). In some embodiments, the peptide comprises the amino acid sequence YQPYPEQEQPILQQ (SEQ ID NO: 17). In some embodiments, the peptide comprises an amino acid sequence of Genbank AAB32025 (8-21) YQPYPEQQQPILQQ (SEQ ID NO: 16) or its partially deamidated homolog Genbank AAB32025 (8-21) [Q15 to E] YQPYPEQEQPILQQ (SEQ ID NO: 17). In some embodiments, the avenin peptide comprises an amino acid sequence of Genbank AAA32714.1 (25-40) EQYQPYPEQQPFMQPL (SEQ ID NO: 18), Genbank AAB23365.1 (3-18) TVQYDPSEQYQPYPEQ (SEQ ID NO: 19), Genbank AAA32716.1 (20-39) TTTVQYNPSEQYQPYPEQQE (SEQ ID NO: 20) or their partially deamidated homologs Genbank AAA32714.1 (25-40) [Q32 to E] EQYQPYPEEQPFMQPL (SEQ ID NO: 21), Genbank AAA32716.1 (20-39)[Q38 to E] TTTVQYNPSEQYQPYPEQEE (SEQ ID NO: 22), Genbank AAB23365.1 (9-24) SEQYQPYPEQQQPFVQ (SEQ ID NO: 23), Genbank Q09097.1 (1-20) TTTVQYDPSEQYQPYPEQQE (SEQ ID NO: 24), Genbank AAB23365.1 (9-24) [Q19 to E] SEQYQPYPEQEQPFVQ (SEQ ID NO: 25), Genbank AAB32025 (7-22) [Q15 to E] QYQPYPEQEQPILQQQ (SEQ ID NO: 27), Genbank AAB32025 (7-22) QYQPYPEQQQPILQQQ (SEQ ID NO: 26), Genbank AAA32715.1 (19-38) AQFDPSEQYQPYPEQQQPIL (SEQ ID NO: 28), Genbank AAB23365.1 (10-29) EQYQPYPEQQQPFVQQQPPF (SEQ ID NO: 29), Genbank Q09097.1 (9-28) [Q19 to E] SEQYQPYPEQEEPFVQQQPP (SEQ ID NO: 32), Genbank P14812.1 (402-421) NNHGQTVFNDILRRGQLLII (SEQ ID NO: 30), Genbank Q09095.1 (2-21) [Q12 and Q18 to E] SEQYQPYPEQEQPFLQEQPL (SEQ ID NO: 33), Genbank AAB23365.1 (10-29) [Q19 to E] EQYQPYPEQEQPFVQQQPPF (SEQ ID NO: 34), Genbank AAB32025 (4-23) [Q15, Q22, and Q23 to E] PSEQYQPYPEQEQPILQQEE (SEQ ID NO: 35), or Genbank Q09095.1 (3-22) EQYQPYPEQQQPFLQQQPLE (SEQ ID NO: 31).

The peptide may be, e.g., less than 50, less than 40, less than 30, or less than 20 amino acids in length. In some embodiments, the peptide comprises an N-terminal glutamate or glutamine that is substituted with a pyroglutamate residue and the C-terminal carboxyl group of the peptide is amidated.

In some embodiments, the composition is a vaccine composition. As used herein, the term “vaccine” refers to a composition comprising peptides that can be administered to a subject having Celiac disease to modulate the subject's response to oats. The vaccine may reduce the immunological reactivity of a subject towards oats. Preferably, the vaccine induces tolerance to oats, allowing oats to be consumed without causing damage to intestinal tissues.

Without being bound by any theory, administration of the vaccine composition to a subject may induce tolerance by clonal deletion of avenin-specific effector T cell populations, for example, avenin-specific CD4⁺T cells, or by inactivation (anergy) of said T cells such that they become less responsive, preferably, unresponsive to subsequent exposure to oats (or peptides thereof). Deletion or inactivation of said T cells can be measured, for example, by contacting ex vivo a sample comprising said T cells with an oat avenin or a peptide thereof and measuring the response of said T cells to the oat avenin or peptide thereof. An exemplary T cell response measurement is measurement of the level of interferon-gamma (IFN-γ) in the sample after contact with the oat avenin or peptide thereof. A decreased level of IFN-γ may indicate deletion or inactivation of said T cells. The level of IFN-γ can be measured using any method known to those of skill in the art, e.g., using immuno-based detection methods such as Western blot or enzyme-linked immunosorbent assay (ELISA).

Alternatively, or in addition, administration of the vaccine composition may modify the cytokine secretion profile of the subject (for example, result in decreased IL-4, IL-2, TNF-α, and/or IFN-γ, and/or increased IL-10). The vaccine composition may induce suppressor T cell subpopulations, for example Treg cells, to produce IL-10 and/or TGF-β and thereby suppress avenin-specific effector T cells. The cytokine secretion profile of the subject can be measured using any method known to those of skill in the art, e.g., using immuno-based detection methods such as Western blot or enzyme-linked immunosorbent assay (ELISA).

There is considerable animal data to support the prophylactic activity of immunodominant peptides for model immune conditions. Accordingly, the vaccine composition of the disclosure can be used for prophylactic treatment of a subject capable of developing sensitivity to oats and/or used in ongoing treatment of a subject who is sensitive to oats. In some embodiments, the composition is for use in treating oats sensitivity in a subject having Celiac disease. In some embodiments, the subject is HLA-DQ2.5 positive. In some embodiments, the subject is HLA-DQ2.5 positive and HLA-DQ8 negative.

### Effective Amount

The amount of a composition to be administered is referred to as the “effective amount”. The term “effective amount” means the amount sufficient to provide the desired therapeutic or physiological effect when administered under appropriate or sufficient conditions.

The effective amounts provided herein are believed to modify the T cell response, e.g., by inducing immune tolerance, to oats in the subject. Thus, a subject treated according to the disclosure preferably is able to eat oats without a significant T cell response which would normally lead to oats sensitivity.

### Pharmaceutically Acceptable Carriers

The composition may include a pharmaceutically acceptable carrier. The term “pharmaceutically acceptable carrier” refers to molecular entities and compositions that do not produce an allergic, toxic or otherwise adverse reaction when administered to a subject, particularly a mammal, and more particularly a human. The pharmaceutically acceptable carrier may be solid or liquid. Useful examples of pharmaceutically acceptable carriers include, but are not limited to, diluents, excipients, solvents, surfactants, suspending agents, buffering agents, lubricating agents, adjuvants, vehicles, emulsifiers, absorbants, dispersion media, coatings, stabilizers, protective colloids, adhesives, thickeners, thixotropic agents, penetration agents, sequestering agents, isotonic and absorption delaying agents that do not affect the activity of the active agents of the disclosure. In some embodiments, the pharmaceutically acceptable carrier is a sodium chloride solution (e.g., sodium chloride 0.9% USP).

The carrier can be any of those conventionally used and is limited only by chemico-physical considerations, such as solubility and lack of reactivity with the active agent, and by the route of administration. Suitable carriers for this disclosure include those conventionally used, for example, water, saline, aqueous dextrose, lactose, Ringer's solution, a buffered solution, hyaluronan, glycols, starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like. Liposomes may also be used as carriers.

Techniques for preparing pharmaceutical compositions are generally known in the art as exemplified by Remington's Pharmaceutical Sciences, 16th Ed. Mack Publishing Company, 1980.

The actual amount administered (or dose or dosage) and the rate and time-course of administration will depend on the nature and severity of the condition being treated as well as the characteristics of the subject to be treated (weight, age, etc.). Prescription of treatment, for example, decisions on dosage, timing, frequency, etc., is within the responsibility of general practitioners or specialists (including human medical practitioner, veterinarian or medical scientist) and typically takes account of the disorder to be treated, the condition of the subject, the site of delivery, the method of administration and other factors known to practitioners. Examples of techniques and protocols can be found in, e.g., Remington's Pharmaceutical Sciences, 16th Ed. Mack Publishing Company, 1980 and Remington: The Science and Practice of Pharmacy, 21st Ed. Lippincott Williams & Wilkins, 2005. Effective amounts may be measured from ng/kg body weight to g/kg body weight per minute, hour, day, week or month.

Toxicity and therapeutic efficacy of the agent can be determined by standard pharmaceutical procedures in cell cultures or experimental animals by determining the IC50 and the maximal tolerated dose. The data obtained from these cell culture assays and animal studies can be used to formulate a range suitable for humans.

### Dosage

It is especially advantageous to formulate the active in dosage unit form for ease of administration and uniformity of dosage. “Dosage unit form” as used herein refers to physically discrete units suited as unitary dosages for the subject to be treated; each unit containing a predetermined quantity of active agent calculated to produce the desired therapeutic effect in association with the required pharmaceutical carrier. The specification for the dosage unit forms are dictated by and directly dependent on the unique characteristics of the active agent and the particular therapeutic effect to be achieved, and the limitations inherent in the art of compounding such an active agent for the treatment of subjects. Examples of dosage units include sealed ampoules and vials and may be stored in a freeze-dried condition requiring only the addition of the sterile liquid carrier immediately prior to use.

The composition may also be included in a container, pack, or dispenser together with instructions for administration.

### Methods of Treatment

Aspects of the disclosure relate to use of the compositions described herein for treating a subject having, suspected of having or at risk of having oats sensitivity.

As used herein, the terms “treat”, “treating”, and “treatment” include abrogating, inhibiting, slowing, or reversing the progression of a disease or condition, or ameliorating or preventing a clinical symptom of the disease (for example, oats sensitivity). Treatment may include induction of immune tolerance (for example, to avenin or peptides thereof), modification of the cytokine secretion profile of the subject and/or induction of suppressor T cell subpopulations to secrete cytokines. Thus, a subject treated according to the disclosure preferably is able to eat oats without a significant T cell response which would normally lead to oat sensitivity.

### Subjects Having Celiac Disease

In some embodiments, methods described herein comprise identifying a subject as sensitivity to or likely to be sensitive to oats, such as subject who has Celiac disease. Thus, it may be desirable to identify subjects, such as subjects with Celiac disease, who are likely to benefit from the methods and compositions described herein. Any diagnostic method or combinations thereof for Celiac disease is contemplated for identifying such a subject. Exemplary methods include, but is not limited to, intestinal biopsy, serology (measuring the levels of one or more antibodies present in the serum), and genotyping (see, e.g., Husby S, Koletzko S, Korponay-Szabo I R, Mearin M L, Phillips A, Shamir R, Troncone R, Giersiepen K, Branski D, Catassi C et al: European Society for Pediatric Gastroenterology, Hepatology, and Nutrition guidelines for the diagnosis of coeliac disease. J Pediatr Gastroenterol Nutr 2012, 54(1):136-160. AND/OR Rubio-Tapia A, Hill I D, Kelly C P, Calderwood A H, Murray J A. ACG clinical guidelines: diagnosis and management of celiac disease. Am J Gastroenterol 2013; 108:656-76. AND/OR Ludvigsson J F, Leffler D A, Bai J C, Biagi F, Fasano A, Green P H, Hadjivassiliou M, Kaukinen K, Kelly C P, Leonard J N, Lundin K E, Murray J A, Sanders D S, Walker M M, Zingone F, Ciacci C. The Oslo definitions for coeliac disease and related terms. Gut 2012; 62:43-52.).

The presence of serum antibodies can be detected using methods known to those of skill in the art, e.g., by ELISA, histology, cytology, immunofluorescence or western blotting. Such antibodies include, but are not limited to: IgA anti-endomysial antibody (IgA EMA), IgA anti-tissue transglutaminase 2 antibody (IgA tTG), IgA anti-deamidated gliadin peptide antibody (IgA DGP), and IgG anti-deamidated gliadin peptide antibody (IgG DGP). Deamidated gliadin peptide-IgA (DGP-IgA) and deamidated gliadin peptide-IgG (DGP-IgG) can be evaluated with commercial kits (e.g. INV 708760, 704525, and 704520, INOVA Diagnostics, San Diego, Calif.).

Subjects can be tested for the presence of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). Exemplary sequences that encode the DQA and DQB susceptibility alleles include HLA-DQA1*0501 (Genbank accession number: AF515813.1) HLA-DQA1*0505 (AH013295.2), HLA-DQB1*0201 (AY375842.1) or HLA-DQB1*0202 (AY375844.1). Methods of genetic testing are well known in the art (see, e.g., Bunce M, et al. Phototyping: comprehensive DNA typing for HLA-A, B, C, DRB1, DRB3, DRB4, DRBS & DQB1 by PCR with 144 primer mixes utilizing sequence-specific primers (PCR-SSP). Tissue Antigens 46, 355-367 (1995); Olerup O, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256.; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3: e2270). Subjects that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D, Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

### Kits

Another aspect of the disclosure relates to kits. In some embodiments, the kit comprises a composition comprising one or more of any of the peptides described herein.

In some embodiments, further comprises an agent for assessing a T cell response. In some embodiments, the agent is a binding partner for a cytokine indicative of the T cell response. In some embodiments, the kit further comprises an agent that recognizes the binding partner for, for example, IFN-γ.

In some embodiments, the kit further comprises a container for whole blood. In some embodiments, the peptide composition is contained within the container (e.g., dried onto the wall of the container). In some embodiments, the composition is contained within a solution separate from the container, such that the composition may be added to the container after blood collection. In some embodiments, the composition is in lyophilized form in a separate container, such that the composition may be reconstituted and added to the container after blood collection, in some embodiments. In some embodiments, the container further contains an anti-coagulant, such as heparin. In some embodiments, the container is structured to hold a defined volume of blood, e.g., 1 mL or 5 mL. In some embodiments, the container is present in the kit in duplicate or triplicate.

In some embodiments, the kit further comprises a negative control container for whole blood and/or a positive control container for whole blood. The negative control container may be, for example, an empty container or a container containing a non-T cell-activating peptide (e.g., dried onto the wall of the container), such as a non-CD4+-T cell-activating peptide. The positive control container may contain, for example, a mitogen such as PHA-L (e.g., 10 units PHA-L). In some embodiments, the negative control container and/or positive control container are structured to hold a defined volume of blood. In some embodiments, the negative control container and/or positive control container are present in the kit in duplicate or triplicate. In some embodiments, the kit comprises any combination of the components mentioned above.

Any suitable binding partner is contemplated. In some embodiments, the binding partner is any molecule that binds specifically to a cytokine as provided herein. A molecule is said to exhibit “specific binding” if it reacts or associates more frequently, more rapidly, with greater duration and/or with greater affinity with a particular target antigen than it does with alternative targets. As described herein, “binds specifically”, when referring to a protein, means that the molecule is more likely to bind to a portion of or the entirety of a protein to be measured than to a portion of or the entirety of another protein. In some embodiments, the binding partner is an antibody or antigen-binding fragment thereof, such as Fab, F(ab)2, Fv, single chain antibodies, Fab and sFab fragments, F(ab′)2, Fd fragments, scFv, or dAb fragments. Methods for producing antibodies and antigen-binding fragments thereof are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). Binding partners also include other peptide molecules and aptamers that bind specifically. Methods for producing peptide molecules and aptamers are well known in the art (see, e.g., published U.S. Patent Application No. 2009/0075834, U.S. Pat. Nos. 7,435,542, 7,807,351, and 7,239,742). In some embodiments, the binding partner is any molecule that binds specifically to an IFN-γ mRNA. As described herein, “binds specifically to an mRNA” means that the molecule is more likely to bind to a portion of or the entirety of the mRNA to be measured (e.g., by complementary base-pairing) than to a portion of or the entirety of another mRNA or other nucleic acid. In some embodiments, the binding partner that binds specifically to an mRNA is a nucleic acid, e.g., a probe. In some embodiments, the kit further comprises a first and second binding partner for a cytokine provided herein, such an IFN-gamma. In some embodiments, the first and second binding partners are antibodies or antigen binding fragments thereof. In some embodiments, the second binding partner is bound to a surface. The second binding partner may be bound to the surface covalently or non-covalently. The second binding partner may be bound directly to the surface, or may be bound indirectly, e.g., through a linker. Examples of linkers, include, but are not limited to, carbon-containing chains, polyethylene glycol (PEG), nucleic acids, monosaccharide units, and peptides. The surface can be made of any material, e.g., metal, plastic, paper, or any other polymer, or any combination thereof. In some embodiments, the first binding partner is washed over the cytokine bound to the second binding partner (e.g., as in a sandwich ELISA). The first binding partner may comprise a detectable label, or an agent that recognizes the first binding partner (e.g., a secondary antibody) may comprise a detectable label.

Any suitable agent that recognizes a binding partner is contemplated. In some embodiments, the binding partner is any molecule that binds specifically to the binding partner. In some embodiments, the agent is an antibody (e.g., a secondary antibody) or antigen-binding fragment thereof, such as Fab, F(ab)2, Fv, single chain antibodies, Fab and sFab fragments, F(ab′)2, Fd fragments, scFv, or dAb fragments. Agents also include other peptide molecules and aptamers that bind specifically to a binding partner. In some embodiments, the binding partner comprises a biotin moiety and the agent is a composition that binds to the biotin moiety (e.g., an avidin or streptavidin).

In some embodiments, the binding partner and/or the agent comprise a detectable label. Any suitable detectable label is contemplated. Detectable labels include any composition detectable by spectroscopic, photochemical, biochemical, immunochemical, chemical, or other physical means, e.g., an enzyme, a radioactive label, a fluorophore, an electron dense reagent, biotin, digoxigenin, or a hapten. Such detectable labels are well-known in the art are detectable through use of, e.g., an enzyme assay, a chromogenic assay, a luminometric assay, a fluorogenic assay, or a radioimmune assay. The reaction conditions to perform detection of the detectable label depend upon the detection method selected.

In some embodiments, the kit further comprises instructions for performing a method provided herein and/or for detecting a T cell response (e.g., detecting a cytokine indicative of the T cell response) in a sample from a subject. In some embodiments, the instructions include the methods described herein. Instructions can be in any suitable form, e.g., as a printed insert or a label.

### Antibodies

Aspects of the disclosure relate to isolated antibodies specific for any of the peptides or compositions described herein.

An antibody that “specifically binds” to a target or an epitope is a term understood in the art, and methods to determine such specific binding are also known in the art. An antibody “specifically binds” to a target antigen if it binds with greater affinity, avidity, more readily, and/or with greater duration than it binds to other substances. For example, an antibody that specifically binds to a peptide described herein or an epitope therein is an antibody that binds this target antigen with greater affinity, avidity, more readily, and/or with greater duration than it binds to other antigens or other epitopes in the same antigen.

In some embodiments, antibodies described herein have a suitable binding affinity to a peptide as described herein. As used herein, “binding affinity” refers to the apparent association constant or KA. The KA is the reciprocal of the dissociation constant (KD). The antibody described herein may have a binding affinity (KD) of at least 10⁻⁵, 10⁻⁶, 10⁻⁷, 10⁻⁸, 10⁻⁹, 10⁻¹M, or lower. An increased binding affinity corresponds to a decreased KD. Higher affinity binding of an antibody to a first target relative to a second target can be indicated by a higher KA (or a smaller numerical value KD) for binding the first target than the KA (or numerical value KD) for binding the second target. In such cases, the antibody has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in, e.g., TRIS-buffer (50 mM TRIS, 150 mM NaCl, 5 mM CaCl2 at pH7.5). These techniques can be used to measure the concentration of bound binding protein as a function of target protein concentration. The concentration of bound binding protein ([Bound]) is related to the concentration of free target protein ([Free]) and the concentration of binding sites for the binding protein on the target where (N) is the number of binding sites per target molecule by the following equation:

[Bound]=[N][Free]/(Kd+[Free])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Also contemplated herein, is use of a peptide or composition described herein for producing an antibody specific for the peptide or composition.

### Methods for Determining the Presence of a Peptide

Other aspects of the disclosure relate to a method for determining the presence of any of the peptides provided herein in a composition. In some embodiments, the method is for determining whether a food or composition is capable of causing a T cell response in a subject. In some embodiments, the method comprises detecting the presence of any of the oat peptides provided herein, such as those that comprise the amino acid sequence PYPEQEQPI (SEQ ID NO: 12) and/or PYPEQQQPI (SEQ ID NO: 11) in the food or the composition.

This may be performed, e.g., by using a binding assay in which one or more binding partners which bind one or more peptides defined herein in a specific manner is contacted with the food or the composition and the formation of peptide/binding-partner complex(es) is detected and used to ascertain the presence of the peptide(s). Exemplary binding partners are described herein. In some embodiments, the binding partner is an antibody. Any suitable format of binding assay can be used, such as an ELISA. Food samples may first be extracted, optionally diluted and then tested in a binding assay. The composition or food typically comprises material from a plant, such as an oat plant.

Such material may be a plant part, such as a harvested product (for example, a seed). The material may be processed products of the plant material, such as a flour or food that comprises avenin. The processing of food material and testing in suitable binding assays is routine (see for example, Kricka, 1998). The composition or food material may be treated with tTG prior to being contacted with the binding partner.

In some embodiments, the composition or food material is contacted with at least one antibody that is specific for a peptide defined herein in deamidated and/or non-deamidated form. Antibodies directed against the peptides defined herein may be provided in kit form for use in an assay for the detection and/or quantification.

### EXAMPLES

### Example 1

Abbreviations: Celiac Disease (CD), Human leukocyte antigen (HLA), tissue transglutaminase (tTG), T cell clones (TCC)

### Introduction

Celiac disease (CD) is characterized by an inappropriate immune reaction in the gut to dietary gluten in wheat, rye, and barley. Recognition of gluten peptides by CD4⁺ T cells is relevant to the pathogenesis of CD. These T cells are restricted by human leukocyte antigen (HLA)-DQ2 or HLA-DQ8. The disease-causing gluten peptides are characterized by their resistance to digestive proteases and selective deamidation by tissue transglutaminase (tTG), resulting in the introduction of negative charges and strong HLA-DQ2 or DQ8 binding. Lifelong strict gluten free diet is the only current treatment. Many patients are non-compliant to this diet due to the lack of choice and low palatability. Patients would benefit from the addition of oats to their diet, but whether oats are also toxic to CD patients is controversial.

Oats belong to the Aveneae tribe of the Gramineae grass family and are phylogenetically distinct from wheat, rye, and barley, which all belong to the Triticeae tribe of the Gramineae grass family. Avenins comprise 10% of the total oat grain compared to 40-50% prolamin in wheat, barley, and rye, and contain approximately half the proline content of these cereals^{1, 2}. Low proline content may result in higher susceptibility to protease digestion, raising the question of whether avenins are biologically capable of inducing an immune response in CD.

Numerous short and long term feeding studies, indicate pure oats are safe for the majority of CD patients (reviewed in Pulido et al³). Importantly, there is evidence in limited numbers of CD patients that clinical symptoms develop, including villous atrophy, following ingestion of pure oats^{4, 5}. Immunologically, avenin-specific small intestinal T cells have been isolated from CD patients and the epitopes were identified as DQ2.5-ave-1a (PYPEQEEPF, SEQ ID NO: 40) and DQ2.5-ave-1b (PYPEQEQPF, SEQ ID NO: 41)⁴. However, the presence of avenin-specific T cells did not correlate with clinical intolerance to oats. Therefore, it was unclear whether these T cells are truly pathogenic. An in silico analysis by Vader et al showed sequence homology between DQ2.5-ave-1a (PYPEQEEPF, SEQ ID NO: 40) and DQ2.5-ave-1b (PYPEQEQPF, SEQ ID NO: 41) and wheat sequences, and that wheat gluten-reactive T cells cross-reacted with DQ2.5-ave-1b (PYPEQEQPF, SEQ ID NO: 41) in vitro⁶. Collectively these findings suggest that intestinal avenin-specific T cells can be isolated from a subset of CD patients, and that immunogenic avenin peptides share homology with other toxic cereals. Thus, it is possible that cross-reactive T cells specific for wheat, barley, or rye are responsible for avenin responses. However, the biochemical properties of avenin peptides have not been extensively explored, and may explain the inefficient induction of oat-specific T cell responses in CD.

The study herein was performed to determine whether the presence of avenin-specific T cells in only a rare subset of patients could account for clinical oats sensitivity. Oral gluten challenge induces gluten-specific T cells in blood in CD⁷, and the specificity of this response is dependent on the cereal ingested⁸. A comprehensive analysis of T cell responses to oats in CD has not been previously performed. Therefore, the study described below was undertaken to characterize polyclonal avenin-specific T cell responses following oats challenge using avenin peptides libraries for epitope mapping. The redundancy of avenin peptide recognition was assessed by raising T cell clones (TCC) to dominant immuno-stimulatory peptides.

### Materials and Methods

### Subjects and Oral Grain Challenge

Australian Caucasian HLA-DQ2.5⁺ and/or HLA-DQ8⁺ CD subjects were recruited by advertisement in the Coeliac Society of Victoria newsletter and all had biopsy-proven CD conforming to ESPGHAN criteria. Patients had been on strict gluten free diet for at least 4 weeks prior to commencing grain challenge. Patients underwent 3-day grain challenge with oats, barley, or combined wheat, barley, and rye muffins, and 6 days after commencement, blood was collected. Day 0 responses were not observed in the selection of patients tested, as described previously⁸.

Oats challenge consisted of 100 g dry weight daily of one of three commercial brands of oats: one not tested for wheat contamination (Uncle Toby's; Oats #1), and two shown by R5 ELISA to contain less than 20 ppm wheat gluten (Tilquhilie Pudding; Oats #2 and Freedom Food; Oats #3). Barley challenge consisted of pearl barley (Ward McKenzie) cooked into a risotto (150 g dry weight daily) and the wheat challenge consisted of four 50 g slices of wheat bread daily (Baker's Delight white loaf cut to toasting thickness). The combined wheat, barley, and rye challenge consisted of muffins baked with wheat flour (White Wings), barley flour (Four Leaf Milling), and rye flour (Four Leaf Milling; 22-25 g dry weight each daily). A symptom diary based on a x-point Likert scale was completed by participants daily from baseline until day 6.

### Antigens

Two avenin peptide libraries were utilized for epitope screening. These were designed using avenin sequences from GenBank and a customized algorithm, as previously described⁹. The initial library consisted of 199 screening-grade 20 mers including all possible 12 mers (Mimotopes). This library was screened with and without tTG pre-treatment. The second library consisted of additional avenin sequences that were synthesized with glutamate substitution at sites predicted to be deamidated by published algorithms^{10, 11} removing the requirement for tTG treatment. This library contained 369 screening-grade 20 mers, including all possible 12 mers (Pepscan). The second round avenin library contained wild-type and deamidated versions of the most immunogenic sequences (25 high quality 16 mers from Mimotopes). High quality peptides containing previously described immunogenic sequences from wheat, barley, and rye were used to test for cross-reactivity⁸ (Pepscan). High quality hordein and avenin peptides for TCC testing and biochemical studies were ordered from Pepscan, Mimotopes, or GL Biochem.

### IFN-γ ELISpot

Peripheral blood mononuclear cells (PBMC) were isolated from heparinized whole blood using Ficoll-Paque™ Plus density-gradient centrifugation (GE Healthcare). IFN-γ ELISpot (Mabtech) was set up as previously described⁸.

### T Cell Cloning

Isolation of TCC from CD patients was carried out as previously described⁸. Briefly, CFSE-labeled PBMC were incubated with antigen for 7 days in IMDM supplemented with 5% heat-inactivated pooled human serum (PHS), 2 mM GlutaMAX™, 100 μM MEM non-essential amino acids (both from Gibco, Invitrogen), and 50 μM 2-mercaptoethanol (Sigma). Proliferated cells were single-cell sorted into 96-well plates containing IL-2, IL-4, anti-CD3 mAb, irradiated feeder cells (allogeneic PBMC and JY-EBV). TCC were expanded and maintained in IL-2 and IL-4 and tested for specificity by ELISpot with irradiated HLA-matched PBMC as antigen-presenting cells. Expanded clones were tested for HLA-DQ restriction, TCR Vbeta usage, and with lysine scans to work out minimal epitopes. See Table 1 below for TCC and epitope information.

TABLE 1
Peptide/epitope nomenclature and raised T cell
clones. *Assumed T cell epitope only.

| HLA-DQ- | | | Raised T |
|---|---|---|---|
| restricted | | Extended | cell clone |
| epitope | 9-mer core | peptide | (TCC) |
| DQ2.5- | PYPEQEEPF | Genbank | |
| ave-1a | (SEQ ID | Q09097.1 | |
| | NO: 40) | (11-26) | |
| | | [Q19 to E] | |
| | | QYQPYPEQE | |
| | | EPFVQQQ | |
| | | (SEQ ID | |
| | | NO: 47) | |
| DQ2.5- | PYPEQEQPF | Genbank | |
| ave-1b | (SEQ ID | AAB23365.1 | |
| | NO: 41) | (11-26) | |
| | | [Q19 to E] | |
| | | SEQYQPYPE | |
| | | QEQPFV | |
| | | (SEQ ID | |
| | | NO: 48) | |
| DQ2.5- | PYPEQEQPI | Genbank | Patient 2 TCC-01; |
| ave-1c | (SEQ ID | AAB32025 | Patient 2 TCC-02 |
| | NO: 12) | (8-21) | |
| | | [Q15 to E] | |
| | | YQPYPEQEQ | |
| | | PILQQ | |
| | | (SEQ ID | |
| | | NO: 17) | |
| DQ2.5- | PIPEQPQPY | Genbank | Patient 2 TCC-03; |
| hor-3a | (SEQ ID | 1103203A | Patient 6 TCC-01; |
| | NO: 4) | (31-45) | Patient 14 TCC-01 |
| | | [Q32 and | |
| | | Q37 to E] | |
| | | PEQPIPEQPQ | |
| | | PYPQQ | |
| | | (SEQ ID | |
| | | NO: 49) | |
| DQ2.5- | PYPEQPYPY | Genbank | Patient 8 TCC-01 |
| hor-3b | (SEQ ID | CAA60681.1 | |
| | NO: 5) | (32-45) | |
| | | [Q38 to E] | |
| | | QPQPYPEQP | |
| | | QPYPQ | |
| | | (SEQ ID | |
| | | NO: 50) | |
| DQ2.5- | PFPEQPQPY | Genbank | |
| hor-3c* | (SEQ ID | CAA37729.1 | |
| | NO: 6) | (25-47) | |
| | | [Q26 and | |
| | | Q31 to E] | |
| | | PEQPFPEQPQ | |
| | | PPQQP | |
| | | (SEQ ID | |
| | | NO: 51) | |
| DQ2.5- | PFPQPEQPF | Genbank | |
| hor-1 | (SEQ ID | P17991.1 | |
| | NO: 42) | (41-48) | |
| | | [Q46 TO E] | |
| DQ2.5- | PQPEQPFPQ | Genbank | |
| hor-2 | (SEQ ID | P17990.1 | |
| | NO: 43) | (53-61) | |
| | | [Q56 TO E] | |
| DQ2.5- | PFPQPELPY | Genbank | Patient 9 TCC-01 |
| glia-α1a | (SEQ ID | P04722.1 | |
| | NO: 44) | (78-92) | |
| | | [Q85 TO E] | |
| | | LQPFPQPELP | |
| | | YPQPQ | |
| | | (SEQ ID | |
| | | NO: 52) | |
| DQ2.5- | PQPELPYPQ | Genbank | Patient 9 TCC-02 |
| glia-α2 | (SEQ ID | P04722.1 | |
| | NO: 45 | (78-92) | |
| | | [Q85 to E] | |
| | | LQPFPQPELP | |
| | | YPQPQ | |
| | | (SEQ ID | |
| | | NO: 52) | |
| DQ2.5- | PFPQPEQPF | Genbank | Patient 10 TCC-01 |
| glia-ω1 | (SEQ ID | AAG17702 | |
| | NO: 42) | (105-118) | |
| | | [Q111 to E] | |
| | | QPFPQPEQPF | |
| | | PWQP | |
| | | (SEQ ID | |
| | | NO: 53) | |
| DQ2.5- | PQPEQPFPW | Genbank | |
| glia-ω2 | (SEQ ID | AAG17702 | |
| | NO: 46) | (105-118) | |
| | | [Q111 to E] | |
| | | QPFPQPEQPF | |
| | | PWQP | |
| | | (SEQ ID | |
| | | NO: 53) | |

### Results

### Avenin-Specific T Cells are Uncommon

In order to understand in vivo polyclonal immune responses to oats, oral oats challenges were performed on 89 CD patients (84 HLA-DQ2.5⁺, 2 HLA-DQ8⁺, and 3 HLA-DQ2.5⁺/HLA-DQ8⁺). Three commercial varieties of oats were tested (See Materials and Methods; Oats #1 n=22, Oats #2 n=28, and Oats #3 n=39), and some patients underwent repeated challenges. On day 6 T cell responses were measured based on IFN-γ production in response to avenin peptide libraries. Responses were observed in 6/80 discreet HLA-DQ2.5⁺patients, and were largely against peptides containing (or homologous to) the previously published avenin epitopes DQ2.5-ave-1a (PYPEQEEPF, SEQ ID NO: 40) and DQ2.5-ave-1b (PYPEQEQPF, SEQ ID NO: 41)^{4, 6} (Table 2). A homologous peptide QYQPYPEQEQPILQQ (also referred to herein as AAB32025 (7-21) [Q15 to E], SEQ ID NO: 39) was the most immunogenic in 5 patients (Table 2). Deamidation at position 6 of the predicted 9-mer core (Table 2 underlined), in most cases enhanced the response to DQ2.5-ave-1a (PYPEQEEPF, SEQ ID NO: 40), DQ2.5-ave-1b (PYPEQEQPF, SEQ ID NO: 41), and AAB32025 (7-21) [Q15 to E] peptides. The exception was Genbank AAA32714.1 (25-40) EQYQPYPEQQPFMQPL (SEQ ID NO: 18), which lacked the glutamine found in the 9-mer core of the other three sequences. 2/6 oats responders were tested with all three oat varieties over 8 years, and responses were observed to avenin peptides with all three brands against similar peptide sequences with varying magnitude.

TABLE 2
IFN-γ ELISpot responses to avenin peptides in CD patients following 3-day oats
challenge. Numbers represent raw SFU. Color-coding represents significant
SFU as a percentage of maximal peptide SFU (***: >70%, **: 40-70%, *: 20-40%,
\hat{ }: 10-20%).

| | Patient number | | | | | | | | | | | | | | | |
|---|---|---|---|---|---|---|---|---|---|---|---|---|---|---|---|---|
| | P2 | | | P11 | | | P12 | | | P3 | | | P1 | | | P13 |
| Peptide (μg/ml) | 1 | 10 | 100 | 1 | 10 | 100 | 1 | 10 | 100 | 1 | 10 | 100 | 1 | 10 | 100 | 50 |

AAB32025 (7-21 (contains DQ2.5-ave-1c)

| | | | | | | | | | | | | | | | | |
|---|---|---|---|---|---|---|---|---|---|---|---|---|---|---|---|---|
| QYQPYPEQQQ | 2 | 10 | 23** | 2 | 11\hat{ } | 4** | 1 | 3 | 7 | 1 | 25** | 4 | 2 | 11 * | 11* | 2 |
| PILQQQ | | | | | | | | | | | | | | | | |
| (SEQ ID | | | | | | | | | | | | | | | | |
| NO: 26) | | | | | | | | | | | | | | | | |
| QYQPYPEQEQ | 6 | 34** | 38*** | 3 | 41*** | 50*** | 1 | 39** | 31** | 19* | 26** | 18* | 11* | 33*** | 11* | 4 |
| PILQQQ | | | | | | | | | | | | | | | | |
| (SEQ ID | | | | | | | | | | | | | | | | |
| NO: 27) | | | | | | | | | | | | | | | | |

Q09097.1 (9-26) (contains DQ2.5-ave-1a)

| | | | | | | | | | | | | | | | | |
|---|---|---|---|---|---|---|---|---|---|---|---|---|---|---|---|---|
| SEQYQPYPEQ | 3 | 3 | 16* | 0 | 0 | 0 | 1 | 3 | 0 | 8 | 9 | 4 | 3 | 5 | 3 | 9 |
| QEPFVQ | | | | | | | | | | | | | | | | |
| (SEQ ID | | | | | | | | | | | | | | | | |
| NO: 54) | | | | | | | | | | | | | | | | |
| QYQPYPEQEE | 7 | 12* | 20* | 2 | 0 | 0 | 4 | 3 | 2 | 20* | 31** | 2 | 8 | 15** | 5 | 9 |
| PFVQQQ | | | | | | | | | | | | | | | | |
| (SEQ ID | | | | | | | | | | | | | | | | |
| NO: 55) | | | | | | | | | | | | | | | | |

AAB23365.1 (9-26) (contains DQ2.5-ave-1b)

| | | | | | | | | | | | | | | | | |
|---|---|---|---|---|---|---|---|---|---|---|---|---|---|---|---|---|
| SEQYQPYPEQ | 4 | 1 | 16* | 0 | 0 | 0 | 0 | 1 | 4 | 1 | 2 | 4 | 2 | 2 | 4 | 1 |
| QQPFVQ | | | | | | | | | | | | | | | | |
| (SEQ ID | | | | | | | | | | | | | | | | |
| NO: 23) | | | | | | | | | | | | | | | | |
| SEQYQPYPEQ | 5 | 9 | 24** | 0 | 3 | 0 | 2 | 4 | 2 | 4 | 7 | 2 | 24*** | 18** | 10 | 13* |
| EQPFVQ | | | | | | | | | | | | | | | | |
| (SEQ ID | | | | | | | | | | | | | | | | |
| NO: 25) | | | | | | | | | | | | | | | | |

AAA32714.1 (25-40)

| | | | | | | | | | | | | | | | | |
|---|---|---|---|---|---|---|---|---|---|---|---|---|---|---|---|---|
| EQYQPYPEQQ | 0 | 3 | 49*** | 3 | 0 | 54*** | 0 | 2 | 31** | 3 | 18* | 5 | 0 | 0 | 4 | 0 |
| PFMQPL | | | | | | | | | | | | | | | | |
| (SEQ ID | | | | | | | | | | | | | | | | |
| NO: 18) | | | | | | | | | | | | | | | | |
| EQYQPYPEEQ | 2 | 0 | 27** | 0 | 0 | 1 | 0 | 0 | 0 | 0 | 2 | 2 | 1 | 1 | 2 | 0 |
| PFMQPL | | | | | | | | | | | | | | | | |
| (SEQ ID | | | | | | | | | | | | | | | | |
| NO: 21) | | | | | | | | | | | | | | | | |

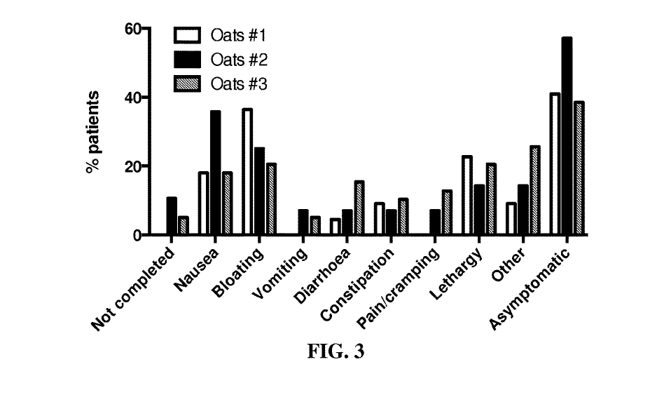

38.5-57.1% of patients were completely asymptomatic following consumption of the three oat varieties (FIG. 3). 5 patients did not complete the challenge; 3 with Oats #2 and 2 with Oats #3. Of the 6 patients that mounted an immune response to avenin, symptoms ranged from severe to asymptomatic. There was no correlation with oats brand, symptoms, or immunogenicity.

### Barley Ingestion Induces Avenin-Specific T Cells

A wheat-specific T cell line specific for DQ2.5-glia-α1a (PFPQPELPY, SEQ ID NO: 44) was shown to cross-react with the avenin epitope DQ2.5-ave-1b (PYPEQEQPF, SEQ ID NO: 41)⁶. In addition, polyclonal T cell responses to selective immuno-dominant wheat peptides were detected in CD patients after consumption of barley and rye⁸. Such cross-reactivity could explain T cell responses to avenin in vivo. In line with this, the most immunogenic avenin sequence AAB32025 (7-21) [Q15 to E] (QYQPYPEQEQPILQQ, SEQ ID NO: 39) contained a possible homolog to the immuno-dominant barley hordein epitope DQ2.5-hor-3a (PIPEQPQPY, SEQ ID NO: 4), recognized by T cells from CD patients only after ingesting barley, but not wheat or rye⁸.

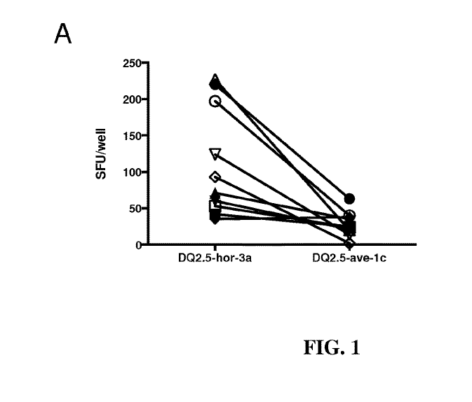

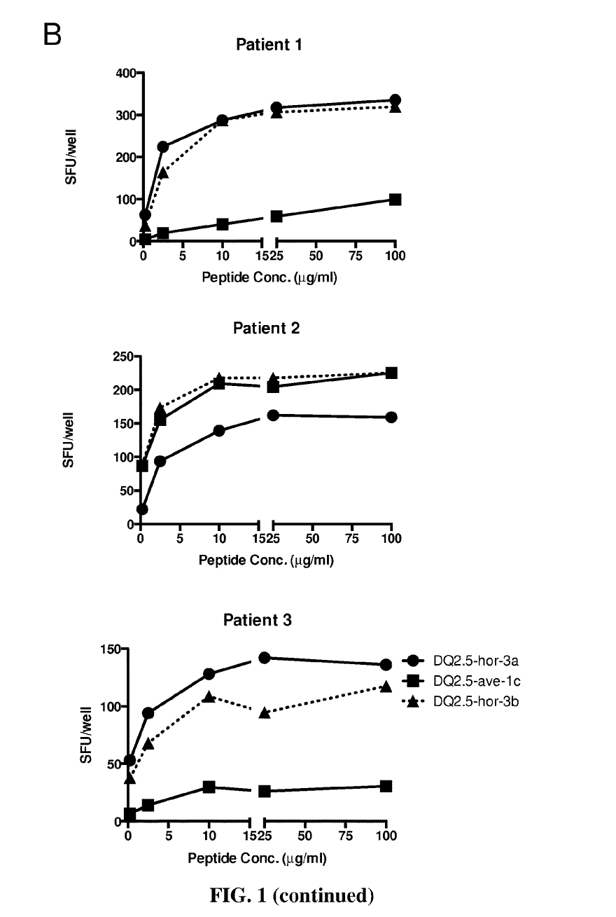

In order to determine if grain cross-reactivity plays a role in avenin-specific immune responses without the bias introduced by feeding any specific gluten containing cereal, a combined wheat, barley, and rye challenge, which did not include oats, was devised. Responses to AAB32025 (7-21) [Q7 to pyroE, Q15 to E, and Q21 to Q-amide] containing the herein referred to as epitope DQ2.5-ave-1c (PYPEQEQPI, SEQ ID NO: 12) and the hordein peptide containing PEQPIPEQPQPYPQ (also referred to herein as 1103203A (31-45) [Q32 and Q37 to E, pyroglutamate at N-terminus and amide group at C-terminus], SEQ ID NO: 56) that encompasses the epitope DQ2.5-hor-3a (PIPEQPQPY, SEQ ID NO: 4) were measured in 19 HLA-DQ2.5⁺ CD patients. 8 patients responded to both, 2 patients responded to the hordein peptide but not the avenin peptide, and 9 patients did not respond to either (FIG. 1a; non responders not shown). 1103203A (31-45) [Q32 and Q37 to E, pyroglutamate at N-terminus and amide group at C-terminus] responses were generally higher than AAB32025 (7-21) [Q7 to pyroE, Q15 to E, and Q21 to Q-amide] responses. Two patients that responded to both peptides following the combined grain challenge and one that responded to 1103203A (31-45) [Q32 and Q37 to E, pyroglutamate at N-terminus and amide group at C-terminus], had also previously been oats challenged with no responses to AAB32025 (7-21) [Q7 to pyroE, Q15 to E, and Q21 to Q-amide].

Due to the high cross-reactivity between 1103203A (31-45) [Q32 and Q37 to E, pyroglutamate at N-terminus and amide group at C-terminus] and AAB32025 (7-21) [Q7 to pyroE, Q15 to E, and Q21 to Q-amide], 3 patients that had previously responded to avenin peptides following oats challenge, were also tested against the oats peptide library and known immunogenic wheat, rye, and barley peptides, following pure barley challenge. All three patients responded to 1103203A (31-45) [Q32 and Q37 to E, pyroglutamate at N-terminus and amide group at C-terminus] and AAB32025 (7-21) [Q7 to pyroE, Q15 to E, and Q21 to Q-amide], and avenin peptides containing homologous sequences to DQ2.5-ave-1a (PYPEQEEPF, SEQ ID NO: 40) and DQ2.5-ave-1b (Table 3). Therefore, barley consumption induces robust T cell responses to avenin peptides in CD patients.

TABLE 3
The most immunogenic peptides from the avenin
peptide library following screening of 3 CD
patients after barley challenge. Numbers
represent raw SFU. Color-coding represents
significant SFU as a percentage of maximal
peptide SFU (***: >70%, **: 40-70%,
*: 20-40%, \hat{ }\hat{ }: 10-20%, \hat{ }: 5-10%).

| | Patient | Patient | Patient |
|---|---|---|---|
| Peptide | 1 | 2 | 3 |
| PEQPIPEQPQPYPQQ 25 μg/ml: | 317*** | 162*** | 142*** |
| 1103203A (31-45) [Q32 and | | | |
| Q37 to E, pyroglutamate at | | | |
| N-terminus and amide | | | |
| group at C-terminus] | | | |
| (SEQ ID NO: 49) | | | |
| YQPYPEQEQPILQQ 25 μg/ml: | 59\hat{ }\hat{ } | 204.5*** | 26\hat{ }\hat{ } |
| AAB32025 (7-21) [Q7 to | | | |
| pyroE, Q15 to E, and | | | |
| Q21 to Q-amide] | | | |
| (SEQ ID NO: 17) | | | |
| Peptide Concentration | 50 | 50 | 50 |
| (μg/ml) | | | |
| SEQYQPYPEQEQPFLQEQPL 50 | 122* | 208*** | 34* |
| μg/ml: Q09095.1 (2-21) | | | |
| [Q12 and Q18 to E] | | | |
| (SEQ ID NO: 33) | | | |
| EQYQPYPEQEQPFVQQQPPF 50 | 99* | 192*** | 14\hat{ } |
| μg/ml: AAB23365.1 | | | |
| (10-29) [Q19 to E] | | | |
| (SEQ ID NO: 34) | | | |
| SEQYQPYPEQEQPFVQ 50 | 77* | 225*** | 36* |
| μg/ml: AAB23365.1 | | | |
| (9-24) [Q19 to E] | | | |
| (SEQ ID NO: 25) | | | |
| QYQPYPEQEQPILQQQ 50 μg/ml: | 58\hat{ }\hat{ } | 220*** | 34* |
| AAB32025 (7-22) [Q15 to E] | | | |
| (SEQ ID NO: 27) | | | |
| AQFDPSEQYQPYPEQQQPIL 50 | 35\hat{ }\hat{ } | 159*** | 24\hat{ }\hat{ } |
| μg/ml: AAA32715.1 (19-38) | | | |
| (SEQ ID NO: 28) | | | |
| PSEQYQPYPEQEQPILQQEE 50 | 27\hat{ } | 159*** | 7 |
| μg/ml: AAB32025 (4-23) | | | |
| [Q15, Q22, and Q23 to E] | | | |
| (SEQ ID NO: 35) | | | |
| EQYQPYPEQQQPFLQQQPLE 50 | 25\hat{ } | 196*** | 24\hat{ }\hat{ } |
| μg/ml: Q09095.1 (3-22) | | | |
| (SEQ ID NO: 31) | | | |
| EQYQPYPEQQPFMQPL 50 μg/ml: | 19\hat{ } | 25\hat{ }\hat{ } | 33* |
| AAA32714.1 (25-40) | | | |
| (SEQ ID NO: 18) | | | |

Responses to avenin peptides did not reach dominance following barley challenge (>70% of the maximum peptide response) except for one patient (Table 3). However, dominant hordein responses were seen in all three patients to peptides containing previously described epitopes DQ2.5-hor-1(PFPQPEQPF, SEQ ID NO: 42), DQ2.5-hor-2 (PQPEQPFPQ, SEQ ID NO: 43), and DQ2.5-hor-3a (PIPEQPQPY, SEQ ID NO: 4), and/or DQ2.5-glia-ω1 (PFPQPEQPF, SEQ ID NO: 42) and DQ2.5-glia-ω2 (PQPEQPFPW, SEQ ID NO: 46)^{6, 8}. Interestingly, 2/3 patients had lower polyclonal T cell responses to AAB32025 (7-21) [Q7 to pyroE, Q15 to E, and Q21 to Q-amide] compared with 1103203A (31-45) [Q32 and Q37 to E, pyroglutamate at N-terminus and amide group at C-terminus] after barley challenge, whereas one patient had a higher response to AAB32025 (7-21) [Q7 to pyroE, Q15 to E, and Q21 to Q-amide]. Three additional patients that responded to AAB32025 (7-21) [Q7 to pyroE, Q15 to E, and Q21 to Q-amide] after barley challenge did not respond to the same peptide following oats challenge. Moreover, six HLA-DQ2.5⁺ CD patients that undertook a wheat challenge did not respond to any avenin peptides, despite 4/6 responding to peptide containing the immuno-dominant DQ2.5-glia-α1a (PFPQPELPY, SEQ ID NO: 44) and DQ2.5-glia-α2 (PQPELPYPY, SEQ ID NO: 57) epitopes. Together this highlights barley and not wheat as the driver of in vivo polyclonal avenin-specific responses, and that avenin responses are induced in a greater number of patients after barley consumption rather than oats.

### Barley and Oats Cross-Reactive T Cells are Present in CD Patients

In order to assess the redundancy of peptide recognition by T cells specific for the dominant immuno-stimulatory peptides AAB32025 (7-21) [Q7 to pyroE, Q15 to E, and Q21 to Q-amide] and 1103203A (31-45) [Q32 and Q37 to E, pyroglutamate at N-terminus and amide group at C-terminus], TCC specific for these peptides were generated and were screened against avenin peptide libraries and known immunogenic wheat, barley, and rye peptides⁸. It was determined that the 1103203A (31-45) [Q32 and Q37 to E, pyroglutamate at N-terminus and amide group at C-terminus] 9-mer core was DQ2.5-hor-3a (PIPEQPQPY, SEQ ID NO: 4) and the AAB32025 (7-21) [Q7 to pyroE, Q15 to E, and Q21 to Q-amide] 9-mer core was PYPEQEQPI (hereafter DQ2.5-ave-1c, using Sollid et al T cell epitope nomenclature, SEQ ID NO: 12¹⁵). The DQ2.5-ave-1c (PYPEQEQPI, SEQ ID NO: 12)-specific TCC (Patient 2 TCC-01) responded to cognate peptide, sequences containing DQ2.5-ave-1b (PYPEQEQPF, SEQ ID NO: 41), and barley peptides, but not peptides including the immuno-dominant DQ2.5-glia-α1a (PFPQPELPY, SEQ ID NO: 44), DQ2.5-glia-α2 (PQPELPYPY, SEQ ID NO: 57), DQ2.5-glia-ω1 (PFPQPEQPF, SEQ ID NO: 42), DQ2.5-glia-ω2 (PQPEQPFPW, SEQ ID NO: 46), or DQ2.5-hor-3a (PIPEQPQPY, SEQ ID NO: 4) (Table 4 and Table 5). The DQ2.5-hor-3a (PIPEQPQPY, SEQ ID NO: 4)-specific TCC (Patient 14 TCC-01 and Patient 6 TCC-01) both responded to cognate peptide and a number of wheat, rye, and barley-derived sequences (Table 4 and Table 5). Patient 6 TCC-01 cross-reacted to DQ2.5-ave-1b (PYPEQEQPF, SEQ ID NO: 41) and one 20 mer peptide containing DQ2.5-ave-1c (PYPEQEQPI, SEQ ID NO: 12), whereas Patient 14 TCC-01 did not cross-react with avenin peptides. All three TCC cross-reacted to the same two barley peptides (Table 4), highlighting the potential for T cell cross-reactivity between oats and barley at a clonal level.

Three TCC specific for wheat epitopes DQ2.5-glia-α1a (PFPQPELPY, SEQ ID NO: 44), DQ2.5-glia-α2 (PQPELPYPY, SEQ ID NO: 57), and DQ2.5-glia-ω1 8 were also tested against the avenin peptide library, but no avenin cross-reactivity was observed (Table 4).

TABLE 4
T cell clones raised to AAB32025 (7-21) [Q7 to pyroE, Q15 to E, and
Q21 to Q-amide] and 1103203A (31-45) [Q32 and Q37 to E, pyroglutamate
at N-terminus and amide group at C-terminus] cross-react with two
hordein peptides whereas wheat-specific T cell clones do not cross-
react. Numbers represent raw SFU. Color-coding represents signifi-
cant SFU as a percentage of maximal peptide SFU (***: >70%,
**: 40-70%, *: 20-40%). Matched T cell clone/antigen is
depicted by matching numbers (e.g , ¹, ², ³).

| | | TCC | TCC | TCC | TCC | TCC | TCC |
|---|---|---|---|---|---|---|---|
| | | Patient | Patient | Patient | Patient | Patient | Patient |
| T cell clone | | 2 | 14 | 6 | 9 | 9 | 10 |
| Peptide | | TCC- | TCC- | TCC- | TCC- | TCC- | TCC- |
| (50 μg/mL) | Sequence | 01¹ | 01² | 01² | 02³ | 01³ | 01⁴ |
| AAB3202 | YQPYPEQE | 300*** | 18 | 2 | 2 | 4 | 5 |
| 5 (7-21) | QPILQQ | | | | | | |
| [Q7 to | (SEQ ID | | | | | | |
| pyroE, | NO: 17) | | | | | | |
| Q15 to E, | | | | | | | |
| and Q21 | | | | | | | |
| to Q- | | | | | | | |
| amide]¹ | | | | | | | |
| 1103203A | PEQPIPEQP | 1 | 48** | 86*** | 2 | 4 | 3 |
| (31-45) | QPYPQQ | | | | | | |
| [Q32 and | (SEQ ID | | | | | | |
| Q37 to E, | NO: 49) | | | | | | |
| pyrogluta- | | | | | | | |
| mate at N- | | | | | | | |
| terminus | | | | | | | |
| and amide | | | | | | | |
| group at | | | | | | | |
| C- | | | | | | | |
| terminus]² | | | | | | | |
| P04722.1 | LQPFPQPE | 0 | 22 | 2 | 240*** | 287*** | 39 |
| (78-92) | LPYPQPQ | | | | | | |
| [Q85 to E, | (SEQ ID | | | | | | |
| N- | NO: 52) | | | | | | |
| terminus | | | | | | | |
| pyroE, C- | | | | | | | |
| amide]³ | | | | | | | |
| AAG1770 | QPFPQPEQ | 2 | 20 | 1 | 0 | 1 | 205*** |
| 2 (105- | PFPWQP | | | | | | |
| 118) | (SEQ ID | | | | | | |
| [Q111 to | NO: 53) | | | | | | |
| E, N- | | | | | | | |
| terminus | | | | | | | |
| pyroE, C- | | | | | | | |
| amide]⁴ | | | | | | | |
| CAA6068 | QPQPYPEQ | 316*** | 27* | 52** | 0 | 1 | 3 |
| 1.1 (32- | PQPYP | | | | | | |
| 45) [Q38 | (SEQ ID | | | | | | |
| to E, N- | NO: 58) | | | | | | |
| terminus | | | | | | | |
| pyroE, C- | | | | | | | |
| amide] | | | | | | | |
| CAA3772 | PEQPFPEQP | 202** | 66*** | 73*** | 0 | 0 | 3 |
| 9.1 (25- | QPYPQQP | | | | | | |
| 47) [Q26 | (SEQ ID | | | | | | |
| and Q31 | NO: 51) | | | | | | |
| to E, C- | | | | | | | |
| amide] | | | | | | | |

TABLE 5
List of Avenin, Hordein, Gliadin, and
Secalin peptides recognized by DQ2.5-
ave-1c (PYPEQEQPI, SEQ ID NO: 12) and
DQ2.5-hor-3a (PIPEQPQPY, SEQ ID NO: 4)-
specific TCC. Raw SFU are shown.
Barley (B), Rye (R), Wheat (W), and
Oats Avenin (A).
*High background, SFU >25 considered as
positive responses.

| | | TCC | TCC | TCC |
|---|---|---|---|---|
| | Sequence | Pa- | Pa- | Pa- |
| | (Genbank accession | tient | tient | tient |
| Grain/ | number, residues | 2 | 6 | 14 |
| code | and modifications) | TCC-01 | TCC-01 | TCC-01 |
| | Mean No Antigen | 1.1 | 2 | 11.3* |
| B04 | FPEQPVPEQPQPYP | 2 | 99 | 78 |
| | 1103203B (30-43) | | | |
| | [Q32 to E, | | | |
| | Q37 to E] | | | |
| | (SEQ ID NO: 59) | | | |
| B06 B08 | FPEQPIPEQPQPYP | 1 | 92 | 66 |
| | 1103203A (30-43) | | | |
| | [Q232 to E, | | | |
| | Q37 to E] | | | |
| | (SEQ ID NO: 60) | | | |
| B12 | PEQPFPEQPQPYPQQP | 202 | 73 | 66 |
| | AFM77740.1 (25-40) | | | |
| | [Q26 to E, | | | |
| | Q31 to E] | | | |
| | (SEQ ID NO: 61) | | | |
| R16 | pEPEQIIPEQPEQPS | 2 | 3 | 59 |
| | AAB58403.1 | | | |
| | (195-208) [Q195 | | | |
| | to pyroglutamate, | | | |
| | Q202 to E, | | | |
| | Q205 to E] | | | |
| | (SEQ ID NO: 62) | | | |
| R06 2 | pEPEQPFPEQPQQII | 0 | 0 | 58 |
| | AAB58403.1 | | | |
| | (187-200) [Q187 | | | |
| | to pyroglutamate, | | | |
| | Q189 to E, | | | |
| | Q194 to E] | | | |
| | (SEQ ID NO: 63) | | | |
| B05 | PQPFPEQPIPEQPQPY | 1 | 60 | 38 |
| | 1103203A (27-42) | | | |
| | [Q32 to E, | | | |
| | Q37 to E] | | | |
| | (SEQ ID NO: 64) | | | |
| B13 | PQPYPEQPQPFPQQPP | 292 | 69 | 12 |
| | 1103203B (39-54) | | | |
| | [Q44 to E] | | | |
| | (SEQ ID NO: 65) | | | |
| W27 | pEQPFTQPEQPTPIQ | 0 | 65 | 13 |
| | AAG17702.1 | | | |
| | (78-91) [Q78 | | | |
| | to pyroglutamate, | | | |
| | Q85 to E] | | | |
| | (SEQ ID NO: 66) | | | |
| B09 | pEQPFPEQPFPEQPQPY | 229 | 64 | 27 |
| | AFM77740.1 | | | |
| | (21-36) [Q21 | | | |
| | to pyroglutamate, | | | |
| | Q26 to E, | | | |
| | Q31 to E] | | | |
| | (SEQ ID NO: 67) | | | |
| B11 | pEQPQPYPEQPQPYP | 316 | 52 | 27 |
| | AFM77740.1 | | | |
| | (31-44) [Q31 | | | |
| | to pyroglutamate, | | | |
| | Q38 to E] | | | |
| | (SEQ ID NO: 68) | | | |
| R03 | pEQPFPQPEQPTPIQ | 1 | 34 | 20 |
| | AAB58403.1 | | | |
| | (65-78) [Q65 | | | |
| | to pyroglutamate, | | | |
| | Q72 to E] | | | |
| | (SEQ ID NO: 69) | | | |
| A03 | EQYQPYPEQEQPFVQQQPPF | 283 | 30 | ND |
| | AAB23365.1 (10-29) | | | |
| | [Q19 to E] | | | |
| | (SEQ ID NO: 34) | | | |
| W17 | pEQPFPQPEQPQLPF | 4 | 23 | 14 |
| | AAK84772.1 | | | |
| | (126-139) [Q126 | | | |
| | to pyroglutamate, | | | |
| | Q133 to E] | | | |
| | (SEQ ID NO: 70) | | | |
| A01 | QPYPEQEQPILEEELLLQQQ | 275 | 16 | ND |
| | CBL51491.1 (22-41) | | | |
| | [Q28 to E, | | | |
| | Q33 to E, | | | |
| | Q34 to E, | | | |
| | Q35 to E] | | | |
| | (SEQ ID NO: 71) | | | |
| A03 | SEQYQPYPEQEQPFVQ | 295 | 12 | 10 |
| | AAB23365.1 (9-24) | | | |
| | [Q19 to E] | | | |
| | (SEQ ID NO: 25) | | | |
| W28 | pEPEQTFPEQPQLPF | 2 | 11 | 33 |
| | AAK84780.1 | | | |
| | (5-18) [Q5 | | | |
| | to pyroglutamate, | | | |
| | Q7 to E, | | | |
| | Q12 to E] | | | |
| | (SEQ ID NO: 72) | | | |
| A01 | QYQPYPEQEQPILQQQ | 288 | 5 | 21 |
| | AAB32025 (7-22) | | | |
| | [Q15 to E] | | | |
| | (SEQ ID NO: 27) | | | |
| W23 2 | pEPFPEQPEQPYPQQ | 150 | 1 | 19 |
| | CAC11056.1 | | | |
| | (53-66) [Q53 | | | |
| | to pyroglutamate, | | | |
| | Q57 to E, | | | |
| | Q60 to E] | | | |
| | (SEQ ID NO: 73) | | | |
| W32 | pEPFPEQPEQPFPQP | 119 | 5 | 14 |
| | ACO40292.1 | | | |
| | (81-94) [Q81 | | | |
| | to pyroglutamate, | | | |
| | Q85 to E, | | | |
| | Q88 to E] | | | |
| | (SEQ ID NO: 74) | | | |
| A04 | SEQYQPYPEQQQPFVQ | 100 | 1 | 11 |
| | AAB23365.1 (9-24) | | | |
| | (SEQ ID NO: 23) | | | |
| A02 | QYQPYPEQQQPILQQQ | 79 | 3 | 22 |
| | AAB32025 (7-22) | | | |
| | (SEQ ID NO: 26) | | | |

Two Hordein Peptides with Homology to DQ2.5-Ave-1c (PYPEQEQPI, SEQ ID NO: 12) and DQ2.5-Hor-3a (PIPEQPQPY, SEQ ID NO: 4) Stimulate an Avenin-Specific T Cell Clone

Interestingly, despite poor cross-reactivity between AAB32025 (7-21) [Q7 to pyroE, Q15 to E, and Q21 to Q-amide] and 1103203A (31-45) [Q32 and Q37 to E, pyroglutamate at N-terminus and amide group at C-terminus] at the clonal level, the TCC tested all cross-reacted with two barley peptides containing 2 homologous sequences to DQ2.5-hor-3a (PIPEQPQPY, SEQ ID NO: 4). These peptides contained near identical predicted 9-mer cores with different amino acids at position 2: PYPEQPQPY (CAA60681.1 (32-45) [Q38 to E, N-terminus pyroE, C-amide], SEQ ID NO: 5) and PFPEQPQPY (CAA37729.1 (25-47) [Q26 and Q31 to E, C-amide], SEQ ID NO: 6). The predicted 9-mer core in CAA60681.1 (32-45) [Q38 to E, N-terminus pyroE, C-amide] only differed from DQ2.5-ave-1b (PYPEQEQPF, SEQ ID NO: 41) and DQ2.5-ave-1c (PYPEQEQPI, SEQ ID NO: 12) by two amino acids (position 6 and 9). Therefore, T cells specific for these homologous peptides could account for the cross-reactivity between oats and barley.

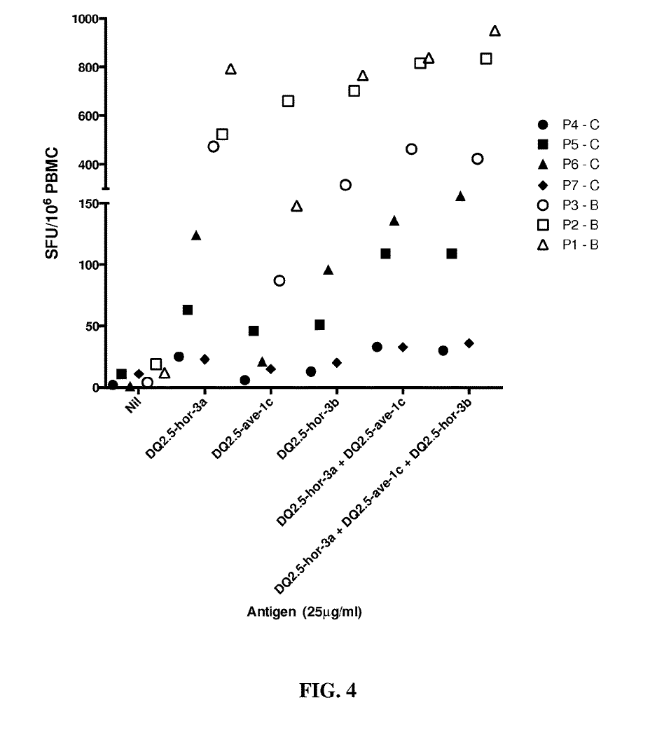

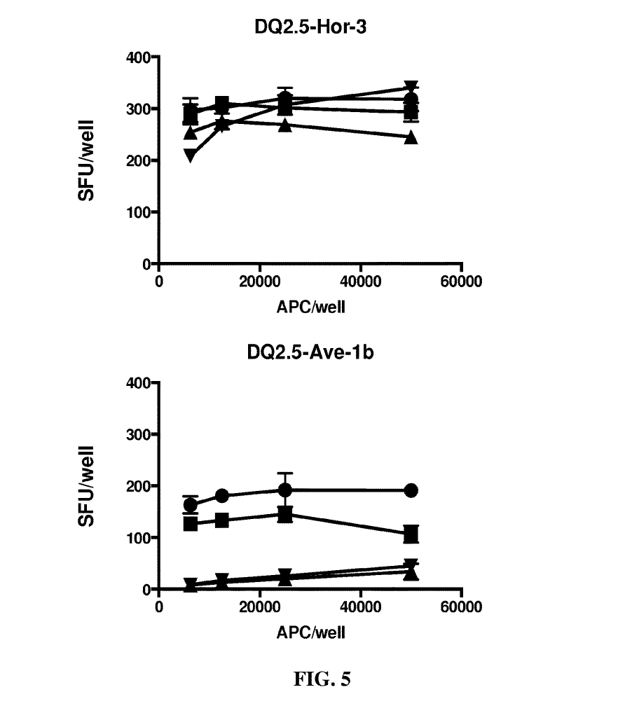

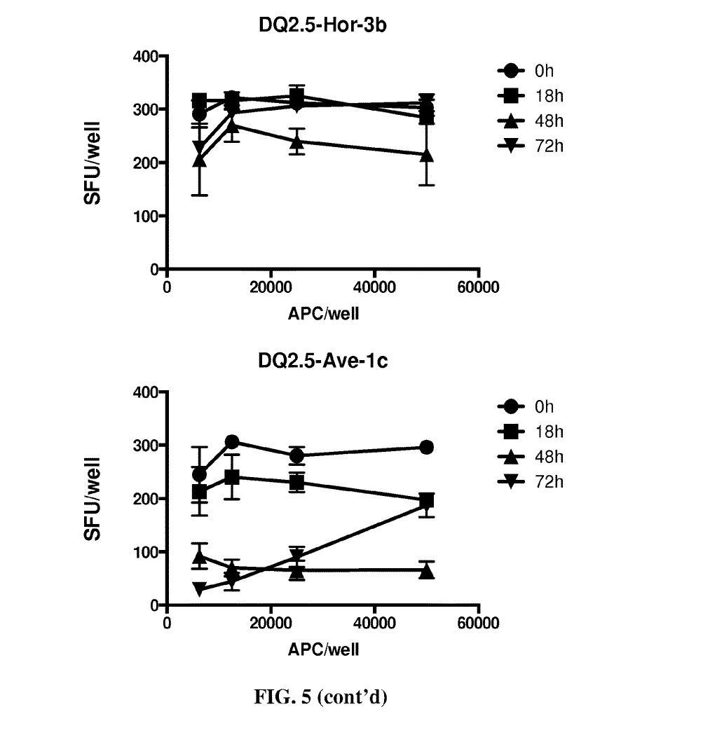

DQ2.5-hor-3a (PIPEQPQPY, SEQ ID NO: 4), CAA60681.1 (32-45) [Q38 to E, N-terminus pyroE, C-amide], and DQ2.5-ave-1c (PYPEQEQPI, SEQ ID NO: 12) responses in 3 oats responders were measured following barley challenge (FIG. 1b). Patients responded to all three peptides to varying degrees. In 2/3 patients, responses to CAA60681.1 (32-45) [Q38 to E, N-terminus pyroE, C-amide] (also referred to herein as DQ2.5-hor-3b) were almost equivalent to DQ2.5-hor-3a (PIPEQPQPY, SEQ ID NO: 4) responses, whilst DQ2.5-ave-1c (PYPEQEQPI, SEQ ID NO: 12) responses were lower (FIG. 1b). In the third patient, responses to CAA60681.1 (32-45) [Q38 to E, N-terminus pyroE, C-amide] were equivalent to DQ2.5-ave-1c (PYPEQEQPI, SEQ ID NO: 12) responses, and DQ2.5-hor-3a (PIPEQPQPY, SEQ ID NO: 4) responses were slightly lower. Moreover, in 4/7 HLA-DQ2.5+ CD patients responses to hordein peptide could account for the response seen to combinations of the three peptides following either barley or combined wheat, barley, and rye challenge (FIG. 4), together suggesting that CAA60681.1 (32-45) [Q38 to E, N-terminus pyroE, C-amide] (or CAA37729.1 (25-47) [Q26 and Q31 to E, C-amide])-specific T cells could explain barley and oats cross-reactivity.

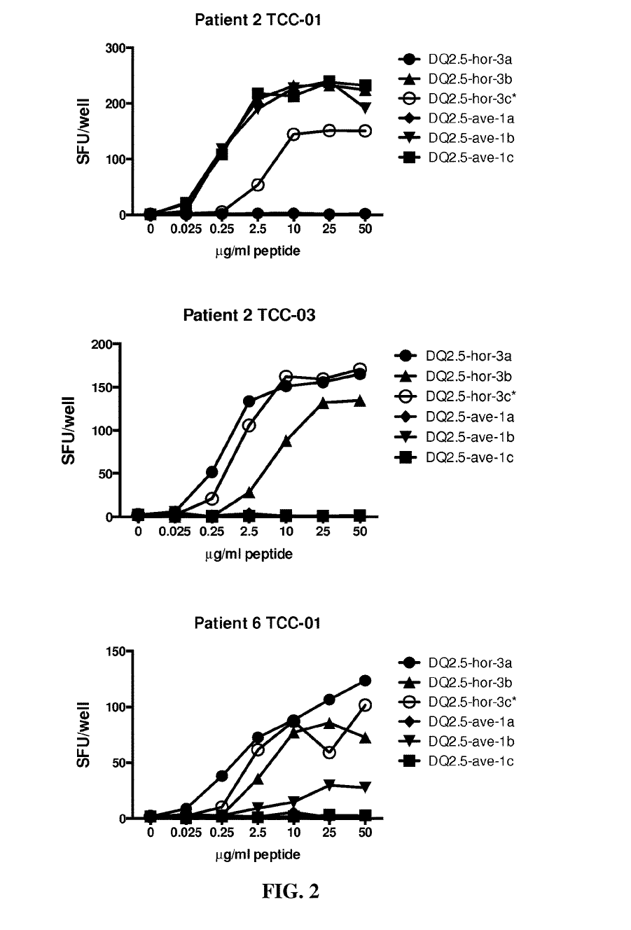

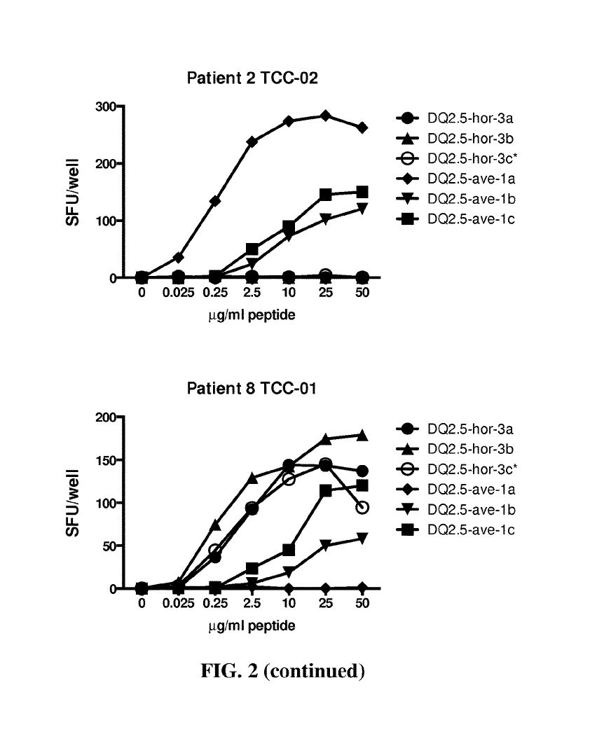

One CAA60681.1 (32-45) [Q38 to E, N-terminus pyroE, C-amide] peptide-specific TCC was generated, and the 9-mer core was verified as PYPEQPQPY (also referred to herein as DQ2.5-hor-3b, SEQ ID NO: 5). Two DQ2.5-ave-1c (PYPEQEQPI, SEQ ID NO: 12) TCC, two DQ2.5-hor-3a (PIPEQPQPY, SEQ ID NO: 4) TCC, and the DQ2.5-hor-3b (PYPEQPQPY, SEQ ID NO: 5) TCC were tested against the homologous peptides: 1103203A (31-45) [Q32 and Q37 to E, pyroglutamate at N-terminus and amide group at C-terminus] (containing DQ2.5-hor-3a), CAA60681.1 (32-45) [Q38 to E, N-terminus pyroE, C-amide] (containing DQ2.5-hor-3b), CAA37729.1 (25-47) [Q26 and Q31 to E, C-amide] containing an epitope referred to as DQ2.5-hor-3c), AAB32025 (7-21) [Q7 to pyroE, Q15 to E, and Q21 to Q-amide] (containing DQ2.5-ave-1c), Q09097.1 (11-26) [Q19 to E] (containing DQ2.5-ave-1a-PYPEQEEPF, SEQ ID NO: 40), and AAB23365.1 (11-26) [Q19 to E] (containing DQ2.5-ave-1bDQ2.5-ave-1b-PYPEQEQPF, SEQ ID NO: 41). Of the two DQ2.5-ave-1c (PYPEQEQPI, SEQ ID NO: 12)-specific TCC, Patient 2 TCC-01 responded to all peptides except for DQ2.5-ave-1a (PYPEQEEPF, SEQ ID NO: 40) and DQ2.5-hor-3a (PIPEQPQPY, SEQ ID NO: 4) (FIG. 2 and Table 6). Patient 2 TCC-02 responded only to oats peptides DQ2.5-ave-1a (PYPEQEEPF, SEQ ID NO: 40), DQ2.5-ave-1b (PYPEQEQPF, SEQ ID NO: 41), and DQ2.5-ave-1c (PYPEQEQPI, SEQ ID NO: 12). The greatest response was to the DQ2.5-ave-1a (PYPEQEEPF, SEQ ID NO: 40) sequence, which contains an additional glutamate in position 7 (FIG. 2). Of the two DQ2.5-hor-3a (PIPEQPQPY, SEQ ID NO: 4)-specific TCC, Patient 6 TCC-01 responded to the three barley peptides, but also DQ2.5-ave-1b (PYPEQEQPF, SEQ ID NO: 41) to a lesser extent (FIG. 2). Patient 2 TCC-03 responded to barley peptides but not oats peptides (FIG. 2). The DQ2.5-hor-3b (PYPEQPQPY, SEQ ID NO: 5)-specific TCC Patient 8 TCC-01 responded to all peptides except for the one containing DQ2.5-ave-1a (PYPEQEEPF, SEQ ID NO: 40), but the lowest responses were to the oats peptides (FIG. 2). 3/5 TCC cross-reacted between grains, all of which recognized DQ2.5-ave-1b (PYPEQEQPF, SEQ ID NO: 41) or DQ2.5-ave-1c (PYPEQEQPI, SEQ ID NO: 12), DQ2.5-hor-3b (PYPEQPQPY, SEQ ID NO: 5), and DQ2.5-hor-3c (PFPEQPQPY, SEQ ID NO: 6). Of three TCC from the oats responding patient 2, one cross-reacted between grains and two did not.

TABLE 6
T cell clone cross-reactivity between
homologous oats and barley peptides.
Number of *s represents significant SFU
as a percentage of maximal peptide SFU
(***: >70%, **: 40-70%, *: 20-40%).
Matched T cell clone/antigen is depicted
by matching numbers (e.g., ¹, ², ³).
\hat{ }Assumed T cell epitope only.

| | | TCC | TCC | TCC | TCC | TCC |
|---|---|---|---|---|---|---|
| | | Pa- | Pa- | Pa- | Pa- | Pa- |
| T cell clone | | tient | tient | tient | tient | tient |
| Peptide | | 2 | 6 | 8 | 2 | 2 |
| (25 | | TCC- | TCC- | TCC- | TCC- | TCC- |
| μg/mL) | Sequence | 03¹ | 01¹ | 01² | 01³ | 02³ |
| DQ2.5- | PIPEQPQPY | 156*** | 107*** | 143*** | 1 | 1 |
| hor-3a¹ | (SEQ ID | | | | | |
| | NO: 4) | | | | | |
| DQ2.5- | PYPEQPQPY | 132*** | 86*** | 175*** | 232*** | 1 |
| hor-3b² | (SEQ ID | | | | | |
| | NO: 5) | | | | | |
| DQ2.5- | PFPEQPQPY | 159*** | 59** | 145*** | 151** | 5 |
| hor-3c\hat{ } | (SEQ ID | | | | | |
| | NO: 6) | | | | | |
| DQ2.5- | PYPEQEEPF | 0 | 1 | 0 | 2 | 284*** |
| ave-1a | (SEQ ID | | | | | |
| | NO: 40) | | | | | |
| DQ2.5- | PYPEQEQPF | 1 | 30* | 50* | 240*** | 102* |
| ave-1b | (SEQ ID | | | | | |
| | NO: 41) | | | | | |
| DQ2.5- | PYPEQEQPI | 1 | 3 | 114** | 238*** | 145** |
| ave-1c³ | (SEQ ID | | | | | |
| | NO: 312) | | | | | |

### Discussion

This is the first comprehensive analysis of relevant CD T cell epitopes contained in oats. These results highlight the scarcity of patients that respond immunologically following 3-day oats challenge, supporting previous studies describing rare clinical symptoms in patients following long-term oats challenge. The T cell epitopes recognized by these rare patients included DQ2.5-ave-1a (PYPEQEEPF, SEQ ID NO: 40), DQ2.5-ave-1b (PYPEQEQPF, SEQ ID NO: 41), DQ2.5-ave-1c (PYPEQEQPI, SEQ ID NO: 12), and the related barley epitopes DQ2.5-hor-3a, DQ2.5-hor-3b (PYPEQPQPY, SEQ ID NO: 5), and DQ2.5-hor-3c (PFPEQPQPY, SEQ ID NO: 6). The study herein describes the first evidence that barley and not just oats induce avenin-specific T cells. T cell cross-reactivity was shown at the clonal level, as barley-specific TCC cross-reacted to avenin peptides. Collectively, these findings support the notion that barley-specific T cells might account for the sensitivity to oats occasionally observed in CD patients.

Oats appear to be well tolerated by most CD patients in feeding studies extending over three-months¹⁷⁻²¹. Feeding studies, particularly long-term ones, are subject to bias from inadvertent ingestion of wheat gluten. Short-term oats challenges of 3 days reduce the time where inadvertent exposure to other toxic grains in patients' diets could confound results. This is the first evidence that oats induce avenin-specific T cells in CD patients. To date avenin T cell responses observed have been limited to T cell lines derived from intestinal biopsies incubated with prolamin.

The immuno-stimulatory avenin peptides identified to date are rich in proline residues. This is consistent with the view that T cell epitopes in gluten cluster in regions with high proline and glutamine content²².

It is believed that the presence of cross-reactive T cells specific for hordein epitopes, which are more favorably recognized by T cells than avenin peptides, could account for toxicity of oats in CD patients. However, a TCC isolated and expanded from an oats sensitive individual Patient 2 only responded to avenin peptides and not hordein. This TCC favored amino acid substitutions in positions 2, 6, and 9 found in DQ2.5-ave-1c (PYPEQEQPI, SEQ ID NO: 12), not in DQ2.5-hor-3a (PIPEQPQPY, SEQ ID NO: 4). From the same individual, a TCC was isolated that responded only to hordein peptides and not avenin, and a TCC was isolated that cross-reacted and responded to both. The presence of solely avenin-specific T cells in this individual could also explain the characteristic that results in oats sensitivity and suggests that high affinity T cells are required to overcome the poor stability of avenin peptides in vivo. Interestingly, this patient had equivalent polyclonal responses to DQ2.5-ave-1c (PYPEQEQPI, SEQ ID NO: 12) and DQ2.5-hor-3b (PYPEQPQPY, SEQ ID NO: 5), in contrast to two other oats sensitive CD patients. It may be relevant that this patient was homozygous for HLA-DQ2, whereas the other two patients were heterozygous. This may suggest that gene dosage may also play a role in overcoming the antigen presentation threshold required for efficient induction of oats-specific T cell responses. Of note, DQ2.5-ave-1c (PYPEQEQPI, SEQ ID NO: 12)-specific TCC were only generated from 1 of 3 oats sensitive patients. An attempt was made to isolate TCC from eight additional patients that responded to the peptide AAB32025 (7-21) [Q7 to pyroE, Q15 to E, and Q21 to Q-amide] following barley challenge, and although proliferation was observed in three following antigen stimulation, these cells could not be expanded or lost specificity. TCC specific for DQ2.5-hor-3a and DQ2.5-hor-3b were generated from two of these patients.

Oats consumption may fail to induce naïve T cells in untreated CD patients due to low bioavailability. The amount of oats that would need to be consumed in order to surpass the threshold of peptides presented to T cells to stimulate an immune response in CD patients would be substantial and unlikely in a normal diet.

The data presented herein provide evidence that barley and not wheat is the major source of avenin-specific T cells. No avenin cross-reactivity by T cell responses induced following oral wheat challenge was observed, or TCC specific for immuno-dominant wheat epitopes DQ2.5-glia-α1a (PFPQPELPY, SEQ ID NO: 44), DQ2.5-glia-α2 (PQPELPYPY, SEQ ID NO: 57), and DQ2.5-glia-ω1. This contrasts to previous data suggesting cross-reactivity between a T cell line specific for DQ2.5-glia-α1a (PFPQPELPY, SEQ ID NO: 44) and the avenin epitope DQ2.5-ave-1b (PYPEQEQPF, SEQ ID NO: 41)⁶.

Lundin et al described an increase in IFN-γ mRNA measured in duodenal biopsies collected following a 12-week oats challenge in 5/18 patients that consented to follow up screening⁵. In 1/5, this also correlated to an increase in Marsh score, demonstrating rare responses to oats. The increase in IFN-γ production could be due to low level inflammation generated by the low number of avenin peptides escaping digestion, which does not reach a great enough level to induce histological damage.

The data herein show that symptoms were not a good indicator for the induction of the immune response, as symptoms did not correlate with the presence of T cell responses in this cohort. The symptoms suffered may be due to the amount of food consumed or the sudden change in diet. Many studies have examined large groups of patients that include oats in their diets, to determine whether oats induce clinical symptoms (reviewed in³). These studies favor the safety and tolerability of oats in the gluten free diet. However, some patients did not tolerate oats and following long-term oats challenge, many patients pulled out of studies due to symptoms suffered and were not followed up.

Although, the different commercial oats induced responses in the same individual CD patient (two tested with all three), the level of response did differ. It is possible that the three brands are derived from different oats cultivars. Recent studies have found some Avena sativa (common oat) varieties to be more immunogenic than others²³⁻²⁵. Mujico et al used a gamma-gliadin TCC that cross-reacted with DQ2.5-ave-1b (PYPEQEQPF, SEQ ID NO: 41) and showed different levels of proliferation induced by 26 different oats cultivars²⁵. In other studies PBMC from children with CD were tested against 3-4 oats cultivars, with differing levels of proliferation and IFN-γ production²⁴. However, this study tested active CD patients still on a gluten containing diet. It has been previously shown that induction of T cells is limited in untreated CD patients and that gluten free diet for at least 2 weeks is required prior to gluten challenge in order to induce robust T cell responses²⁶. For the study herein, information on the oat varieties used for gluten challenge was not able to be obtained. Moreover, due to the lengthy time periods between challenges, inter-assay variation cannot be excluded.

The lack of an immune response in the majority of patients following oats consumption does suggest that pure oats can safely be included in diets of such individuals. The commercial oats #1 were not tested by ELISA for contamination by other gluten-containing grains, something commonly reported for most typical commercial brands²⁷. It is interesting to note that despite the potential for wheat gluten contamination, most patients did not react immunologically to oats #1, or the pure oats #2 and #3.

This study marks the first in vivo analysis of T cell responses to oats in CD and supports a novel explanation for oats toxicity. Based on previous clinical studies and this study, it appears that the majority of patients can safely consume oats as part of their diet with monitoring over time. Grain cross-reactivity was evident based on strong homology of sequences from barley and oats. For this reason it is possible that barley primes high affinity T cells that will later respond to oats. The redundancy of avenin peptide recognition by T cells specific for barley supports the notion that a minimal list of dominant peptides encompassing wheat, rye and barley, but not oats, is sufficient to encompass the immune response to all forms of gluten in CD.

### REFERENCES

1. Cornell H, Wieser H, Belitz H D. Characterization of the gliadin-derived peptides which are biologically active in coeliac disease. Clinica Chimica Acta 1992; 213:37-50.
2. Wieser H. The precipitating factor in coeliac disease. Baillieres Clinical Gastroenterology 1995; 9:191-207.
3. Pulido O M, Gillespie Z, Zarkadas M, et al. Introduction of oats in the diet of individuals with celiac disease: a systematic review. Adv Food Nutr Res 2009; 57:235-85.
4. Arentz-Hansen H, Fleckenstein B, Molberg O, et al. The molecular basis for oat intolerance in patients with celiac disease. PLoS Med 2004; 1:e1.
5. Lundin K E, Nilsen E M, Scott H G, et al. Oats induced villous atrophy in coeliac disease. Gut 2003; 52:1649-52.
6. Vader L W, Stepniak D T, Bunnik E M, et al. Characterization of cereal toxicity for celiac disease patients based on protein homology in grains.[see comment]. Gastroenterology 2003; 125:1105-13.
7. Anderson R P, Degano P, Godkin A J, et al. In vivo antigen challenge in celiac disease identifies a single transglutaminase-modified peptide as the dominant A-gliadin T-cell epitope. Nature Medicine 2000; 6:337-42.
8. Tye-Din J A, Stewart J A, Dromey J A, et al. Comprehensive, quantitative mapping of T cell epitopes in gluten in celiac disease. Sci Transl Med 2010; 2:41ra51.
9. Beissbarth T, Tye-Din J A, Smyth G K, et al. A systematic approach for comprehensive T-cell epitope discovery using peptide libraries. Bioinformatics 2005; 21 Suppl 1:i29-37.
10. Fleckenstein B, Molberg O, Qiao S W, et al. Gliadin T cell epitope selection by tissue transglutaminase in celiac disease. Role of enzyme specificity and pH influence on the transamidation versus deamidation process. Journal of Biological Chemistry 2002; 277:34109-16.
11. Vader L W, de Ru A, van der Wal Y, et al. Specificity of tissue transglutaminase explains cereal toxicity in celiac disease. Journal of Experimental Medicine 2002; 195:643-9.
12. Webb A I, Dunstone M A, Williamson N A, et al. T cell determinants incorporating beta-amino acid residues are protease resistant and remain immunogenic in vivo. J Immunol 2005; 175:3810-8.
13. Fallang L E, Bergseng E, Hotta K, et al. Differences in the risk of celiac disease associated with HLA-DQ2.5 or HLA-DQ2.2 are related to sustained gluten antigen presentation. Nat Immunol 2009; 10:1096-101.
14. Lanzavecchia A, Reid P A, Watts C. Irreversible association of peptides with class II MHC molecules in living cells. Nature 1992; 357:249-52.
15. Sollid L M, Qiao S W, Anderson R P, et al. Nomenclature and listing of celiac disease relevant gluten T-cell epitopes restricted by HLA-DQ molecules. Immunogenetics 2012; 64:455-60.
16. Shan L, Qiao S W, Arentz-Hansen H, et al. Identification and analysis of multivalent proteolytically resistant peptides from gluten: implications for celiac sprue. J Proteome Res 2005; 4:1732-41.
17. Hoffenberg E J, Haas J, Drescher A, et al. A trial of oats in children with newly diagnosed celiac disease. Journal of Pediatrics 2000; 137:361-6.
18. Janatuinen E K, Pikkarainen P H, Kemppainen T A, et al. A comparison of diets with and without oats in adults with celiac disease.[see comment]. New England Journal of Medicine 1995; 333:1033-7.
19. Janatuinen E K, Kemppainen T A, Julkunen R J, et al. No harm from five year ingestion of oats in coeliac disease.[see comment]. Gut 2002; 50:332-5.
20. Srinivasan U, Leonard N, Jones E, et al. Absence of oats toxicity in adult coeliac disease. BMJ 1996; 313:1300-1.
21. Storsrud S, Olsson M, Arvidsson Lenner R, et al. Adult coeliac patients do tolerate large amounts of oats. European Journal of Clinical Nutrition 2003; 57:163-9.
22. Arentz-Hansen H, McAdam S N, Molberg O, et al. Celiac lesion T cells recognize epitopes that cluster in regions of gliadins rich in proline residues.[see comment]. Gastroenterology 2002; 123:803-9.
23. Silano M, Di Benedetto R, Maialetti F, et al. Avenins from different cultivars of oats elicit response by coeliac peripheral lymphocytes. Scand J Gastroenterol 2007; 42:1302-5.
24. Comino I, Real A, de Lorenzo L, et al. Diversity in oat potential immunogenicity: basis for the selection of oat varieties with no toxicity in coeliac disease. Gut 2011; 60:915-22.
25. Mujico J R, Mitea C, Gilissen L J W J, et al. Natural variation in avenin epitopes among oat varieties: Implications for celiac disease. Journal of Cereal Science 2011; 54:8-12.
26. Anderson R P, van Heel D A, Tye-Din J A, et al. T cells in peripheral blood after gluten challenge in coeliac disease. Gut 2005; 54:1217-23.
27. Hernando A, Mujico J R, Mena M C, et al. Measurement of wheat gluten and barley hordeins in contaminated oats from Europe, the United States and Canada by Sandwich R5 ELISA. Eur J Gastroenterol Hepatol 2008; 20:545-54.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for identifying a subject as sensitive to or likely sensitive to oats, the method comprising:
a) determining a T cell response to a barley peptide in a sample comprising a T cell from the subject; and
b) identifying the subject as
(i) sensitive to or likely sensitive to oats if the T cell response to the barley peptide is elevated compared to a control T cell response, or
(ii) not sensitive to or likely not sensitive to oats if the T cell response to the barley peptide is reduced compared to the control T cell response or the same as the control T cell response.

2. The method of claim 1, wherein the step of determining comprises contacting the sample with a composition comprising the barley peptide and measuring a T cell response to the barley peptide.

3. The method of claim 2, wherein measuring a T cell response to the barley peptide comprises measuring a level of IFN-gamma in the sample.

4. The method of claim 3, wherein measuring comprises an enzyme-linked immunosorbent assay (ELISA) or an enzyme-linked immunosorbent spot (ELISpot) assay.

5. The method of any one of claims 1 to 4, wherein the barley peptide is a hordein peptide.

6. The method of claim 5, wherein the hordein peptide comprises the amino acid sequence PIPQQPQPY (SEQ ID NO: 1), PYPQQPQPY (SEQ ID NO: 2), PFPQQPQPY (SEQ ID NO: 3), PIPEQPQPY (SEQ ID NO: 4), PYPEQPQPY (SEQ ID NO: 5), PFPEQPQPY (SEQ ID NO: 6), PIPDQPQPY (SEQ ID NO: 7), PYPDQPQPY (SEQ ID NO: 8), or PFPDQPQPY (SEQ ID NO: 9).

7. The method of claim 6, wherein the hordein peptide comprises the amino acid sequence PIPQQPQPY (SEQ ID NO: 1), PIPEQPQPY (SEQ ID NO: 4) or PIPDQPQPY (SEQ ID NO: 7).

8. The method of any one of claims 1 to 7, wherein the subject has Celiac disease.

9. The method of any one of the preceding claims, wherein the method further comprises performing an additional test if the subject is identified as sensitive to or likely sensitive to oats.

10. The method of claim 9, wherein the additional test comprises measuring a T cell response to an oat peptide.

11. The method of claim 10, wherein the oat peptide comprises the amino acid sequence PYPEQQQPI (SEQ ID NO: 11), PYPEQEQPI (SEQ ID NO: 12), PYPEQDQPI (SEQ ID NO: 13), PYPDQEQPI (SEQ ID NO: 14) or PYPDQDQPI (SEQ ID NO: 15).

12. The method of claim 10, wherein the oat peptide comprises: the amino acid sequence of Genbank AAB32025 (8-21) YQPYPEQQQPILQQ (SEQ ID NO: 16) or its partially deamidated homolog Genbank AAB32025 (8-21) [Q15 to E] YQPYPEQEQPILQQ (SEQ ID NO: 17); the amino acid sequence of Genbank AAA32714.1 (25-40) EQYQPYPEQQPFMQPL (SEQ ID NO: 18), Genbank AAB23365.1 (3-18) TVQYDPSEQYQPYPEQ (SEQ ID NO: 19) or Genbank AAA32716.1 (20-39) TTTVQYNPSEQYQPYPEQQE (SEQ ID NO: 20) or the partially deamidated homolog Genbank AAA32714.1 (25-40) [Q32 to E] EQYQPYPEEQPFMQPL (SEQ ID NO: 21) or Genbank AAA32716.1 (20-39)[Q38 to E] TTTVQYNPSEQYQPYPEQEE (SEQ ID NO: 22); the amino acid sequence of Genbank AAB23365.1 (9-24) SEQYQPYPEQQQPFVQ (SEQ ID NO: 23) or Genbank Q09097.1 (1-20) TTTVQYDPSEQYQPYPEQQE (SEQ ID NO: 24) or the partially deamidated homolog Genbank AAB23365.1 (9-24) [Q19 to E] SEQYQPYPEQEQPFVQ (SEQ ID NO: 25); the amino acid sequence of Genbank AAB32025 (7-22) QYQPYPEQQQPILQQQ (SEQ ID NO: 26) or its partially deamidated homolog Genbank AAB32025 (7-22) [Q15 to E] QYQPYPEQEQPILQQQ (SEQ ID NO: 27); the amino acid sequence of Genbank AAA32715.1 (19-38) AQFDPSEQYQPYPEQQQPIL (SEQ ID NO: 28), Genbank AAB23365.1 (10-29) EQYQPYPEQQQPFVQQQPPF (SEQ ID NO: 29), Genbank P14812.1 (402-421) NNHGQTVFNDILRRGQLLII (SEQ ID NO: 30) or Genbank Q09095.1 (3-22) EQYQPYPEQQQPFLQQQPLE (SEQ ID NO: 31) or the partially deamidated Genbank Q09097.1 (9-28) [Q19 to E] SEQYQPYPEQEEPFVQQQPP (SEQ ID NO: 32), Genbank Q09095.1 (2-21) [Q12 and Q18 to E] SEQYQPYPEQEQPFLQEQPL (SEQ ID NO: 33), Genbank AAB23365.1 (10-29) [Q19 to E] EQYQPYPEQEQPFVQQQPPF (SEQ ID NO: 34) or Genbank AAB32025 (4-23) [Q15, Q22, and Q23 to E] PSEQYQPYPEQEQPILQQEE (SEQ ID NO: 35).

13. The method of any one of the preceding claims, wherein the sample comprises whole blood or peripheral blood mononuclear cells.

14. The method of any one of the preceding claims, wherein the method further comprises administering a composition comprising barley or oats, or a peptide thereof, to the subject prior to determining the T cell response.

15. The method of claim 14, wherein the composition comprising barley or oats, or a peptide thereof, is administered to the subject more than once prior to determining the T cell response.

16. The method of claim 14 or 15, wherein the composition comprising barley or oats, or a peptide thereof, is a foodstuff.

17. The method of any one of the preceding claims, wherein the method further comprises treating the subject if identified as sensitive or likely sensitive to oats or providing information to the subject about a treatment.

18. The method of any one of the preceding claims, where the method further comprises a step of recommending an oats-free diet if the subject is identified as sensitive to or likely sensitive to oats or providing information to the subject about such a diet.

19. A method for identifying a subject as sensitive to or likely sensitive to oats, the method comprising:
a) determining a T cell response to a peptide comprising the amino acid sequence PYPEQQQPI (SEQ ID NO: 11), PYPEQEQPI (SEQ ID NO: 12), PYPEQDQPI (SEQ ID NO: 13), PYPDQEQPI (SEQ ID NO: 14) or PYPDQDQPI (SEQ ID NO: 15) in a sample comprising a T cell from the subject; and
b) identifying the subject as
(i) sensitive to or likely sensitive to oats if the T cell response to the peptide is elevated compared to a control T cell response, or
(ii) not sensitive to or likely not sensitive to oats if the T cell response to the peptide is reduced compared to the control T cell response or the same as the control T cell response.

20. The method of claim 19, wherein the step of determining comprises contacting the sample with a composition comprising the peptide and measuring a T cell response to the peptide.

21. The method of claim 20, wherein measuring a T cell response to the peptide comprises measuring a level of IFN-gamma in the sample.

22. The method of claim 21, wherein measuring comprises an enzyme-linked immunosorbent assay (ELISA) or an enzyme-linked immunosorbent spot (ELISpot) assay.

23. The method of any one of claims 19 to 22, wherein the subject has Celiac disease.

24. The method of any one of claims 19 to 23, wherein the sample comprises whole blood or peripheral blood mononuclear cells.

25. The method of any one of claims 19 to 24, wherein the method further comprises administering a composition comprising barley or oats, or a peptide thereof, to the subject prior to determining the T cell response.

26. The method of claim 25, wherein the composition comprising barley or oats, or a peptide thereof, is administered to the subject more than once prior to determining the T cell response.

27. The method of claim 25 or 26, wherein the composition comprising barley or oats, or a peptide thereof, is a foodstuff.

28. The method of any one of claims 19 to 27, wherein the method further comprises treating the subject if identified as sensitive or likely sensitive to oats or providing information to the subject about a treatment.

29. The method of any one of claims 19 to 28, where the method further comprises a step of recommending an oats-free diet if the subject is identified as sensitive to or likely sensitive to oats or providing information to the subject about such a diet.

30. A composition comprising a peptide comprising the amino acid sequence PYPEQEQPI (SEQ ID NO: 12), PYPEQDQPI (SEQ ID NO: 13), PYPDQEQPI (SEQ ID NO: 14) or PYPDQDQPI (SEQ ID NO: 15).

31. The composition of claim 30, wherein the peptide is less than 50 amino acids in length.

32. The composition of claim 31, wherein the peptide is less than 30 amino acids in length.

33. The composition of any one of claims 30-32, wherein the peptide comprises the amino acid sequence YQPYPEQEQPILQQ (SEQ ID NO: 17), YQPYPEQDQPILQQ (SEQ ID NO: 36), YQPYPDQEQPILQQ (SEQ ID NO: 37) or YQPYPDQDQPILQQ (SEQ ID NO: 38).

34. A vaccine composition comprising the composition of any one of claims 30-33.

35. A kit comprising the composition of any one of claims 30-33.

36. The kit of claim 35, wherein the kit comprises a container for whole blood.

37. The kit of claim 36, wherein the container is a vial or tube.

38. The kit of claim 36 or 37, wherein the composition is dried on the wall of the container for whole blood.

39. The kit of any of claims 35-38, wherein the composition is in solution or lyophilized in a separate container.

40. The kit of any of claims 35-39, wherein the kit further comprises a negative control container.

41. The kit of claim 40, wherein the negative control container is a vial or tube.

42. The kit of any of claims 35-41, wherein the kit further comprises a positive control container.

43. The kit of claim 42, wherein the positive control container is a vial or tube.

44. The kit of any of claims 35-43, wherein the negative and/or positive control container(s) are present in duplicate or triplicate.

45. A method of modulating a T cell response to an oat peptide in a subject who is sensitive to oats, the method comprising administering to the subject an effective amount of a composition comprising a peptide comprising the amino acid sequence PYPEQQQPI (SEQ ID NO: 11), PYPEQEQPI (SEQ ID NO: 12), PYPEQDQPI (SEQ ID NO: 13), PYPDQEQPI (SEQ ID NO: 14) or PYPDQDQPI (SEQ ID NO: 15).

46. The method of claim 45, wherein the peptide is less than 50 amino acids in length.

47. The method of claim 46, wherein the peptide is less than 30 amino acids in length.

48. The method of any one of claims 45 to 47, wherein the peptide comprises the amino acid sequence YQPYPEQQQPILQQ (SEQ ID NO: 16), YQPYPEQEQPILQQ (SEQ ID NO: 17), YQPYPEQDQPILQQ (SEQ ID NO: 36), YQPYPDQEQPILQQ (SEQ ID NO: 37) or YQPYPDQDQPILQQ (SEQ ID NO: 38).

49. A method, comprising:
detecting the presence of the amino acid sequence PYPEQEQPI (SEQ ID NO: 12) and/or PYPEQQQPI (SEQ ID NO: 11) in a composition.

50. The method of claim 49, wherein the method is for determining whether the composition is capable of causing a T cell response in a subject.

51. The method of claim 49 or 50, wherein the composition is a foodstuff.

52. A composition comprising a peptide comprising: the amino acid sequence of Genbank AAB32025 (8-21) YQPYPEQQQPILQQ (SEQ ID NO: 16) or its partially deamidated homolog Genbank AAB32025 (8-21) [Q15 to E] YQPYPEQEQPILQQ (SEQ ID NO: 17); the amino acid sequence of Genbank AAA32714.1 (25-40) EQYQPYPEQQPFMQPL (SEQ ID NO: 18), Genbank AAB23365.1 (3-18) TVQYDPSEQYQPYPEQ (SEQ ID NO: 19) or Genbank AAA32716.1 (20-39) TTTVQYNPSEQYQPYPEQQE (SEQ ID NO: 20) or the partially deamidated homolog Genbank AAA32714.1 (25-40) [Q32 to E] EQYQPYPEEQPFMQPL (SEQ ID NO: 21) or Genbank AAA32716.1 (20-39)[Q38 to E] TTTVQYNPSEQYQPYPEQEE (SEQ ID NO: 22); the amino acid sequence of Genbank AAB23365.1 (9-24) SEQYQPYPEQQQPFVQ (SEQ ID NO: 23) or Genbank Q09097.1 (1-20) TTTVQYDPSEQYQPYPEQQE (SEQ ID NO: 24) or the partially deamidated homolog Genbank AAB23365.1 (9-24) [Q19 to E] SEQYQPYPEQEQPFVQ (SEQ ID NO: 25); the amino acid sequence of Genbank AAB32025 (7-22) QYQPYPEQQQPILQQQ (SEQ ID NO: 26) or the partially deamidated homolog Genbank AAB32025 (7-22) [Q15 to E] QYQPYPEQEQPILQQQ (SEQ ID NO: 27); the amino acid sequence of Genbank AAA32715.1 (19-38) AQFDPSEQYQPYPEQQQPIL (SEQ ID NO: 28), Genbank AAB23365.1 (10-29) EQYQPYPEQQQPFVQQQPPF (SEQ ID NO: 29), Genbank P14812.1 (402-421) NNHGQTVFNDILRRGQLLII (SEQ ID NO: 30) or Genbank Q09095.1 (3-22) EQYQPYPEQQQPFLQQQPLE (SEQ ID NO: 31) or the partially deamidated Genbank Q09097.1 (9-28) [Q19 to E] SEQYQPYPEQEEPFVQQQPP (SEQ ID NO: 32), Genbank Q09095.1 (2-21) [Q12 and Q18 to E] SEQYQPYPEQEQPFLQEQPL (SEQ ID NO: 33), Genbank AAB23365.1 (10-29) [Q19 to E] EQYQPYPEQEQPFVQQQPPF (SEQ ID NO: 34) or Genbank AAB32025 (4-23) [Q15, Q22, and Q23 to E] PSEQYQPYPEQEQPILQQEE (SEQ ID NO: 35).

53. The composition of claim 52, wherein the peptide is less than 50 amino acids in length.

54. The composition of claim 53, wherein the peptide is less than 30 amino acids in length.

55. An antibody that specifically binds to the peptide of the composition of any of the preceding claims.

56. Use of the peptide of the composition of any of the preceding claims for producing an antibody that specifically binds to the peptide.


SEQUENCE LISTING (74 sequences)

SEQ ID NO: 1 - protein, 9 aa, Artificial Sequence
  Synthetic peptide
PIPQQPQPY

SEQ ID NO: 2 - protein, 9 aa, Artificial Sequence
  Synthetic peptide
PYPQQPQPY

SEQ ID NO: 3 - protein, 9 aa, Artificial Sequence
  Synthetic peptide
PFPQQPQPY

SEQ ID NO: 4 - protein, 9 aa, Artificial Sequence
  Synthetic peptide
PIPEQPQPY

SEQ ID NO: 5 - protein, 9 aa, Artificial Sequence
  Synthetic peptide
PYPEQPQPY

SEQ ID NO: 6 - protein, 9 aa, Artificial Sequence
  Synthetic peptide
PFPEQPQPY

SEQ ID NO: 7 - protein, 9 aa, Artificial Sequence
  Synthetic peptide
PIPDQPQPY

SEQ ID NO: 8 - protein, 9 aa, Artificial Sequence
  Synthetic peptide
PYPDQPQPY

SEQ ID NO: 9 - protein, 9 aa, Artificial Sequence
  Synthetic peptide
PFPDQPQPY

SEQ ID NO: 10 - protein, 9 aa, Artificial Sequence
  Synthetic peptide
PYPEQPQPF

SEQ ID NO: 11 - protein, 9 aa, Artificial Sequence
  Synthetic peptide
PYPEQQQPI

SEQ ID NO: 12 - protein, 9 aa, Artificial Sequence
  Synthetic peptide
PYPEQEQPI

SEQ ID NO: 13 - protein, 9 aa, Artificial Sequence
  Synthetic peptide
PYPEQDQPI

SEQ ID NO: 14 - protein, 9 aa, Artificial Sequence
  Synthetic peptide
PYPDQEQPI

SEQ ID NO: 15 - protein, 9 aa, Artificial Sequence
  Synthetic peptide
PYPDQDQPI

SEQ ID NO: 16 - protein, 14 aa, Artificial Sequence
  Synthetic peptide
YQPYPEQQQPILQQ

SEQ ID NO: 17 - protein, 14 aa, Artificial Sequence
  Synthetic peptide
YQPYPEQEQPILQQ

SEQ ID NO: 18 - protein, 16 aa, Artificial Sequence
  Synthetic peptide
EQYQPYPEQQPFMQPL

SEQ ID NO: 19 - protein, 16 aa, Artificial Sequence
  Synthetic peptide
TVQYDPSEQYQPYPEQ

SEQ ID NO: 20 - protein, 20 aa, Artificial Sequence
  Synthetic peptide
TTTVQYNPSEQYQPYPEQQE

SEQ ID NO: 21 - protein, 16 aa, Artificial Sequence
  Synthetic peptide
EQYQPYPEEQPFMQPL

SEQ ID NO: 22 - protein, 20 aa, Artificial Sequence
  Synthetic peptide
TTTVQYNPSEQYQPYPEQEE

SEQ ID NO: 23 - protein, 16 aa, Artificial Sequence
  Synthetic peptide
SEQYQPYPEQQQPFVQ

SEQ ID NO: 24 - protein, 20 aa, Artificial Sequence
  Synthetic peptide
TTTVQYDPSEQYQPYPEQQE

SEQ ID NO: 25 - protein, 16 aa, Artificial Sequence
  Synthetic peptide
SEQYQPYPEQEQPFVQ

SEQ ID NO: 26 - protein, 16 aa, Artificial Sequence
  Synthetic peptide
QYQPYPEQQQPILQQQ

SEQ ID NO: 27 - protein, 16 aa, Artificial Sequence
  Synthetic peptide
QYQPYPEQEQPILQQQ

SEQ ID NO: 28 - protein, 20 aa, Artificial Sequence
  Synthetic peptide
AQFDPSEQYQPYPEQQQPIL

SEQ ID NO: 29 - protein, 20 aa, Artificial Sequence
  Synthetic peptide
EQYQPYPEQQQPFVQQQPPF

SEQ ID NO: 30 - protein, 20 aa, Artificial Sequence
  Synthetic peptide
NNHGQTVFNDILRRGQLLII

SEQ ID NO: 31 - protein, 20 aa, Artificial Sequence
  Synthetic peptide
EQYQPYPEQQQPFLQQQPLE

SEQ ID NO: 32 - protein, 20 aa, Artificial Sequence
  Synthetic peptide
SEQYQPYPEQEEPFVQQQPP

SEQ ID NO: 33 - protein, 20 aa, Artificial Sequence
  Synthetic peptide
SEQYQPYPEQEQPFLQEQPL

SEQ ID NO: 34 - protein, 20 aa, Artificial Sequence
  Synthetic peptide
EQYQPYPEQEQPFVQQQPPF

SEQ ID NO: 35 - protein, 20 aa, Artificial Sequence
  Synthetic peptide
PSEQYQPYPEQEQPILQQEE

SEQ ID NO: 36 - protein, 14 aa, Artificial Sequence
  Synthetic peptide
YQPYPEQDQPILQQ

SEQ ID NO: 37 - protein, 14 aa, Artificial Sequence
  Synthetic peptide
YQPYPDQEQPILQQ

SEQ ID NO: 38 - protein, 14 aa, Artificial Sequence
  Synthetic peptide
YQPYPDQDQPILQQ

SEQ ID NO: 39 - protein, 15 aa, Artificial Sequence
  Synthetic peptide
QYQPYPEQEQPILQQ

SEQ ID NO: 40 - protein, 9 aa, Artificial Sequence
  Synthetic peptide
PYPEQEEPF

SEQ ID NO: 41 - protein, 9 aa, Artificial Sequence
  Synthetic peptide
PYPEQEQPF

SEQ ID NO: 42 - protein, 9 aa, Artificial Sequence
  Synthetic peptide
PFPQPEQPF

SEQ ID NO: 43 - protein, 9 aa, Artificial Sequence
  Synthetic peptide
PQPEQPFPQ

SEQ ID NO: 44 - protein, 9 aa, Artificial Sequence
  Synthetic peptide
PFPQPELPY

SEQ ID NO: 45 - protein, 9 aa, Artificial Sequence
  Synthetic peptide
PQPELPYPQ

SEQ ID NO: 46 - protein, 9 aa, Artificial Sequence
  Synthetic peptide
PQPEQPFPW

SEQ ID NO: 47 - protein, 16 aa, Artificial Sequence
  Synthetic peptide
QYQPYPEQEEPFVQQQ

SEQ ID NO: 48 - protein, 15 aa, Artificial Sequence
  Synthetic peptide
SEQYQPYPEQEQPFV

SEQ ID NO: 49 - protein, 15 aa, Artificial Sequence
  Synthetic peptide
PEQPIPEQPQPYPQQ

SEQ ID NO: 50 - protein, 14 aa, Artificial Sequence
  Synthetic peptide
QPQPYPEQPQPYPQ

SEQ ID NO: 51 - protein, 16 aa, Artificial Sequence
  Synthetic peptide
PEQPFPEQPQPYPQQP

SEQ ID NO: 52 - protein, 15 aa, Artificial Sequence
  Synthetic peptide
LQPFPQPELPYPQPQ

SEQ ID NO: 53 - protein, 14 aa, Artificial Sequence
  Synthetic peptide
QPFPQPEQPFPWQP

SEQ ID NO: 54 - protein, 16 aa, Artificial Sequence
  Synthetic peptide
SEQYQPYPEQQEPFVQ

SEQ ID NO: 55 - protein, 16 aa, Artificial Sequence
  Synthetic peptide
QYQPYPEQEEPFVQQQ

SEQ ID NO: 56 - protein, 14 aa, Artificial Sequence
  Synthetic peptide
PEQPIPEQPQPYPQ

SEQ ID NO: 57 - protein, 9 aa, Artificial Sequence
  Synthetic peptide
PQPELPYPY

SEQ ID NO: 58 - protein, 13 aa, Artificial Sequence
  Synthetic peptide
QPQPYPEQPQPYP

SEQ ID NO: 59 - protein, 14 aa, Artificial Sequence
  Synthetic peptide
FPEQPVPEQPQPYP

SEQ ID NO: 60 - protein, 14 aa, Artificial Sequence
  Synthetic peptide
FPEQPIPEQPQPYP

SEQ ID NO: 61 - protein, 16 aa, Artificial Sequence
  Synthetic peptide
PEQPFPEQPQPYPQQP

SEQ ID NO: 62 - protein, 14 aa, Artificial Sequence
  Synthetic peptide
EPEQIIPEQPEQPS

SEQ ID NO: 63 - protein, 14 aa, Artificial Sequence
  Synthetic peptide
EPEQPFPEQPQQII

SEQ ID NO: 64 - protein, 16 aa, Artificial Sequence
  Synthetic peptide
PQPFPEQPIPEQPQPY

SEQ ID NO: 65 - protein, 16 aa, Artificial Sequence
  Synthetic peptide
PQPYPEQPQPFPQQPP

SEQ ID NO: 66 - protein, 14 aa, Artificial Sequence
  Synthetic peptide
EQPFTQPEQPTPIQ

SEQ ID NO: 67 - protein, 16 aa, Artificial Sequence
  Synthetic peptide
EQPFPEQPFPEQPQPY

SEQ ID NO: 68 - protein, 14 aa, Artificial Sequence
  Synthetic peptide
EQPQPYPEQPQPYP

SEQ ID NO: 69 - protein, 14 aa, Artificial Sequence
  Synthetic peptide
EQPFPQPEQPTPIQ

SEQ ID NO: 70 - protein, 14 aa, Artificial Sequence
  Synthetic peptide
EQPFPQPEQPQLPF

SEQ ID NO: 71 - protein, 20 aa, Artificial Sequence
  Synthetic peptide
QPYPEQEQPILEEELLLQQQ

SEQ ID NO: 72 - protein, 14 aa, Artificial Sequence
  Synthetic peptide
EPEQTFPEQPQLPF

SEQ ID NO: 73 - protein, 14 aa, Artificial Sequence
  Synthetic peptide
EPFPEQPEQPYPQQ

SEQ ID NO: 74 - protein, 14 aa, Artificial Sequence
  Synthetic peptide
EPFPEQPEQPFPQP